(19) United States
(12) Patent Application Publication
(10) Pub. No.: US 2005/0203578 A1
(43) Pub. Date: Sep. 15, 2005

(54) Process and apparatus for treating biological organisms

(76) Inventors: Michael L. Weiner, Webster, NY (US); Robert W. Gray, Rochester, NY (US); Stuart G. MacDonald, Pultneyville, NY (US); Jack A. Tuszynski, Edmonton (CA); Kendrick Goss, Brighton, MA (US); Amara D. Angelica, Webster, NY (US)

Correspondence Address: HOWARD J. GREENWALD P.C., 349 W. COMMERCIAL STREET SUITE 2490, EAST ROCHESTER, NY, 14445-2408, US

(21) Appl. No.: 11/066,418

(22) Filed: Feb. 25, 2005

Related U.S. Application Data
(63) Continuation-in-part of application No. 09/930,364, filed on Aug. 15, 2001, now abandoned.

(51) Int. Cl. A61N 1/00; H03C 3/06; H03C 3/09

(52) U.S. Cl. 607/2; 332/128

(57) ABSTRACT

An apparatus for treating a biological organism comprising a device for emitting and delivering energy to the biological organism, a programmable controller for varying the type and amount of energy emitted, and apparatus for sensing a condition of the biological organism.

### CROSS-REFERENCE TO RELATED PATENT APPLICATIONS

This application is a continuation-in-part of applicants' U.S. patent application Ser. No. 09/930,364, filed on Aug. 15, 2001.

### FIELD OF THE INVENTION

This invention relates to treatment biological organisms with various forms of energy, particularly electromagnetic energy.

### BACKGROUND OF THE INVENTION

The application of exterior photonic and other electromagnetic energy to a body for therapeutic purposes is well known. Thus, for example, U.S. Pat. No. 5,843,074 discloses “An improved non-coherent pulsed and colored light stimulation device used for therapeutic effects in living creatures.” Similarly, U.S. Pat. No. 5,500,009 discloses “A method of treating herpes by illuminating a herpes affected dermal zone with continuous wave (CW) non-coherent radiation emitted by at least one light emitting diode (LED), the radiation having a narrow bandwidth centered at a wavelength suitable for herpes treatment, and maintaining the light radiation for a prescribed treatment duration.” The entire disclosure of each of these United States patents is hereby incorporated by reference into this specification.

Chinese and other Eastern medical traditions have mapped out acupuncture points over the body. Other traditions have mapped out “meridian” and “chakra” points of the body. Devices have been developed to locate and measure (see U.S. Pat. Nos. 4,408,617 and 4,016,870) and stimulate (see U.S. Pat. Nos. 6,113,530 and 4,535,784) such “biologically active” points using light and/or other electromagnetic and/or vibrational and/or heat energies. The entire disclosure of each of these United States patents is hereby incorporated by reference into this specification.

It is known that the application of high frequency electromagnetic signals can have beneficial therapeutic effects on tissues. Thus, e.g., U.S. Pat. No. 6,246,912 discloses “A method and apparatus are provided for altering a function of tissue in a patient.” The tissue affected can include that of the brain, as is disclosed in U.S. Pat. No. 5,983,141 (“Method and apparatus for altering neural tissue function”), which discloses “A method and apparatus for altering a function of neural tissue in a patient. An electromagnetic signal is applied to the neural tissue through an electrode.” The entire disclosure of each of these United States patents is hereby incorporated by reference into this specification.

It is known that the application of extremely low frequency (less than 100 hertz) electromagnetic signals can have beneficial therapeutic effects. See, for example, the paper “Therapeutic aspects of electromagnetic fields for soft-tissue healing” by B. F. Siskin and J. Walker, 1995 published in Electromagnetic fields: biological interactions and mechanisms, M. Blank editor, Advances in Chemistry Series 250, American Chemical Society, Washington D.C., pages 277-285.

Millimeter waves have wavelengths of from about 1 to about 10 millimeters, corresponding to frequencies of from about 300 to about 30 gigahertz. In recent years, a substantial amount of research has been conducted regarding the biological and medical effects of such millimeter waves. See, e.g., an article by A. G. Pakhomov et al. entitled “Current state and implications of research on biological effects of millimeter waves: A review of the literature,” published in 1998 in Bioelectromagnetics, 19(7), at pages 393-413.

Today millimeter wave therapy, also known as “extremely high frequency therapy,” has become an approved and accepted method of medical treatment in Russia and many former Soviet republics. More than 2,000 physicians from all over Russia have completed formal education courses in Moscow on the medical uses of millimeter waves; the method is currently used in more than 1,500 hospitals and clinics in the Russian Federation; more than 1,000,000 patients have undergone this treatment; and more than 10,000 millimeter wave devices have been sold to research and clinical institutions. See, e.g., a paper by A. Yu. Lebedeva entitled “Millimeter waves in clinical practice in Russia: a Review” that was presented on Oct. 31, 2000 in Zvenigorod, Russia at the 12^{th} Russian Symposium on Millimeter Waves in Medicine and Biology.

It has been determined that low intensity millimeter waves (with power levels of less than about 11 milliwatts per square centimeter) have effects on cell growth and proliferation, activity of enzymes, the function of excitable membranes, peripheral receptors, and other biological systems. See, e.g., the aforementioned 1998 article by A. G. Pakhomov et al. It has also been determined that, in animals and humans, local millimeter wave exposure has stimulated tissue repair and regeneration, alleviated stress reactions, and facilitated recovery in a wide range of diseases. See, e.g., an 1999 article by N. N. Lebedeva and T. I. Kotorovskaya entitled “Experimental and clinical studies in the field of biological effects of millimeter waves” (review, part 1) published in Russian in Millimetrovye Volny v. Biologii I Meditsine (“Millimeter Waves in Medicine and Biology”), 3(15), pages 3-14.

Millimeter wave generators are well known to those skilled in the art and are commercially available. Thus, e.g., referring to U.S. Pat. No. 3,596,695, the entire disclosure of which is hereby incorporated by reference into this specification, it is disclosed that “Referring now to FIG. 1, there is illustrated in block form an apparatus embodying the present invention. The apparatus of FIG. 1 includes a variable microwave generator 10. The microwave generator 10 is continuously variable over a predetermined frequency range as indicated by the arrow 11. Such microwave generators are readily obtainable in the trade. For example, Model No. 440XXH represents a series of microwave generators obtainable from Hughes Aircraft Company. By way of example, Model No. 44076H is a millimeter wave generator having a 3 milliwatt output over a 10 gigahertz bandwidth between 60 to 90 gigahertz and includes an isolator. Other models are available with other frequency ranges and with similar power outputs.”

U.S. Pat. No. 6,101,015 discloses a microwave or millimeter wave generator. U.S. Pat. No. 5,777,572 discloses a gyrotron oscillator millimeter wave generator for producing high power millimeter wave beams for jamming and/or damaging electronic equipment; the generator of this patent produces 20 millisecond megawatt pulses at a frequency of from 100 to 140 gigahertz. U.S. Pat. No. 5,760,397 discloses a millimeter wave imaging system. U.S. Pat. No. 5,507,791 discloses a millimeter wave generator producing radiation with a frequency of from 40 to 70 gigahertz. U.S. Pat. No. 5,379,309 discloses a photonic down conversion system which employs a millimeter wave generator. In FIG. 3 (element 15) of U.S. Pat. No. 5,344,099, a millimeter wave generator is shown. U.S. Pat. No. 5,227,800 discloses a millimeter wave generator used to illuminate objects in the field of view of a millimeter wave camera. A millimeter wave generator is mentioned in claim 16 of U.S. Pat. No. 5,223,352. U.S. Pat. No. 5,152,286 discloses a spark (noise) generator for producing extremely high frequency (EHF) electromagnetic radiation. U.S. Pat. No. 5,131,409 discloses a microwave resonance therapy generator. U.S. Pat. No. 4,306,174 discloses a radio wave generator for ultra-high frequencies. U.S. Pat. No. 4,286,230 discloses a near millimeter wave generator with a dielectric cavity. The entire disclosure of each of these United States patents is hereby incorporated by reference into this specification.

Millimeter wave generators, devices incorporating them, and processing using them, are described in many different Russian patents. Reference may be had, e.g., to Russian patents 2122395 (method for treatment of auditory nerve neuritis), 2089166 (device for extremely high frequency therapy).

In a book entitled Light: Medicine of the Future, Bear and Company, Santa Fe, N. Mex., 1991, Jacob Liberman discussed the therapeutic effects of light for treating, e.g., cholesterol, cortisone, stress, cancer, venereal disease, viral infection, tuberculoses, etc. Reference also may be had, e.g., to U.S. Pat. No. 5,454,837.

The application of acoustic energy is also known to have therapeutic effects on the body and its tissues and organs. Thus, e.g., U.S. Pat. No. 5,687,729 discloses “A source of therapeutic acoustic waves for minimally invasive treatment of internal body regions with the therapeutic acoustic waves has a number of source parts which emit the acoustic waves.” U.S. Pat. No. 5,458,130 discloses “Non-invasive therapeutic treatment and/or quantitative evaluation of musculoskeletal tissue are performed in vivo by subjecting musculoskeletal tissue to an ultrasonic acoustic signal pulse of finite duration, and involving a composite sine-wave signal consisting of plural discrete frequencies that are spaced in the ultrasonic region to approximately 2 megahertz the excitation signal is repeated substantially in the range 1 to 1000 Hz.” U.S. Pat. No. 5,209,221 discloses “A device for generating sonic signal forms for limiting, preventing or regressing the growth of pathological tissue comprises an ultrasonic transmission system for transmitting sound waves, focused on the tissue to be treated, by way of a coupling medium.” The entire disclosure of each of these United States patents is hereby incorporated by reference into this specification.

Bone material may also be treated using electromagnetic and/or vibrational energies. Thus, e.g., pulsing electromagnetic fields are widely used by orthopedic physicians to stimulate the healing of fracture non-unions. See, e.g., the 1995 article by CAL Bassett entitled “Bioelectromagnetics in the service of medicine” published in Electromagnet Fields: Biological Interactions and Mechanisms, M. Blank editor, Advances in Chemistry Series 250, American Chemical Society, Washington D.C., pp. 261-275. U.S. Pat. No. 5,309,898 discloses “Non-invasive therapeutic treatment and/or quantitative evaluation of bone tissue are performed in vivo, by subjecting bone to an ultrasonic acoustic signal pulse of finite duration, and involving a composite sine-wave signal consisting of plural discrete frequencies that are spaced in the ultrasonic region to approximately 2 MHz; the excitation signal is repeated substantially in the range 1 to 1000 Hz.” The entire disclosure of each of these United States patents is hereby incorporated by reference into this specification.

The application of acoustic energy to a biological system can produce an electromagnetic response. Applying both acoustic and electromagnetic energy at the same time has therapeutic effects on the body. International patent publication WO015097A2 discloses “The present invention makes use of resonant acoustic and/or acousto-EM energy applied to inorganic or biologic structures for the detection and/or identification, and for augmentation and/or disruption of function within the biologic structure.” The entire disclosure of this patent is hereby incorporated by reference into this specification.

Implantable medical devices are now commonplace. For example, U.S. Pat. No. 6,212,063 discloses “An implantable medical device such as a defibrillator is described.” Another example is U.S. Pat. No. 6,143,035, which discloses “An implanted piezoelectric module generates charge which may be applied to tissue or used to power or recharge an implanted device such as a pump or pacemaker.” The entire disclosure of each of these United States patents is hereby incorporated by reference into this specification.

It is known that the application of certain electromagnetic energies and signals can change the biological effectiveness of fluids including water. References to such effects include Dr. Alan Halls' book Water, Electricity and Health, Hawthorn Press, 1997 and references cited therein, as well as the papers “Digital Recording/Transmission of the Cholinergic Signal” by Dr. J. Benveniste, et. al. and references therein. Another reference is the 1987 article by R. V. S. Choy, J. A. Monro, and C. W. Smith, “Electrical sensitivities in allergy patients” published in Clinical Ecology IV(3):93-102, which states “A protocol for clinical testing has been devised based on the confrontation-neutralization technique for chemical allergens. Neutralizing frequencies can usually be found and magnetic fields at these frequencies can be used to “potentize” water for therapeutic purposes. In a given patient, the symptoms provoked electrically are similar to those provoked chemically and those provoked by the patient's environment. Electrical and chemical stimuli and neutralization appear to be interchangeable.” Hence treatment of water and other bodily fluids could be included into existing internal or external devices which sample the bodily fluids. For example, insulin pumps, kidney machines, flow cytometers, and syringes.

Means are also available for sensing or predicting pathological disturbances or imbalances in physiological parameters. In some cases these sensors are useful in following changes in parameters during the course of treatments.

Transmural electrical potential differences have been suggested as an early marker for the detection of colon cancer. See the 1986 article by D A. Goller, W. F. Weidema, and R. J. Davies entitled “Transmural electrical potential difference as an early marker in colon cancer” published in Archives of Surgery 121:345-350. Surface electrical potentials have been tested in the diagnosis of breast lesions. See the 1994 article by B. A. Weiss, G. A. P. Ganepola, H. P. Freeman, Y-S Hsu, and M. L. Faupel entitled “Surface electrical potentials as a new modality in the diagnosis of breast lesions—A preliminary survey” published in Breast Diseases 7:91-98). Transcranial magnetic stimulation has been used to evaluate the probable outcome of patients post-stroke. See the 2000 article by U. Ziemann entitled “Transcranial magnetic stimulation: Its current role in the evaluation of patients post-stroke” published in Neurology Report 24(3):82-93.

The vulnerability of the heart to ventricular arrhythmias and sudden cardiac death has been correlated with certain patterns in the electrocardiogram known as T-wave alternans. Noninvasive techniques are available that permit the accurate measurement in ambulatory patients. U.S. Pat. No. 5,560,368 discloses methodology for automated QT variability measurement to determine risk of malignant arrhythmias, that involves sensing fluctuations in voltage resulting from electrical activity of a heart and assessing changes in QT interval for each heartbeat using the entire T wave. U.S. Pat. No. 5,555,888 discloses a method for automatic, adaptive assessment of myocardial electrical instability to assess the patient's likelihood for myocardial electrical instability. The entire disclosure of each of these United States patents is hereby incorporated by reference into this specification.

Optical approaches to the non-invasive measurement of blood glucose are disclosed by R. W. Waynant and V. M. Chenault in an 1998 article entitled “Overview of non-invasive fluid glucose measurement using optical techniques to maintain glucose control in diabetes mellitus” published in IEEE Lasers and Electro-Optics Society Proceedings 12:2. Reference also may bed had to a 1998 article by C. Marwick entitled “Development of noninvasive methods to monitor blood glucose levels in people with diabetes” published in the Journal of the American Medical Association 280(4):312-313. U.S. Pat. No. 5,989,409 discloses a method for measuring the concentration of glucose diffused from a source to a working electrode which assembly includes a scavenging electrode. U.S. Pat. No. 6,233,471 discloses a method for continually or continuously measuring the concentration of target chemical analytes present in a biological system, and processing analyte-specific signals to obtain a measurement value that is closely correlated with the concentration of the target chemical analyte in the biological system. One important application of the invention involves a method for signal processing in a system for monitoring blood glucose values. The entire disclosure of each of these United States patents is hereby incorporated by reference into this specification.

Electromagnetic probes can be used to monitor microvascular changes taking place in response to diabetes, as is disclosed by A. S. De Vriese, J. Van de Voorde, J. J. Blom, P. M. Vanhoutte, M. Verbeke, and N. H. Lameire in the 2000 article entitled “The impaired renal vasodilator response attributed to endothelium-derived hyperpolarizing factor in streptozotocin—induced diabetic rats is restored by 5-methyltetrahydrofolate” published in Diabetologia 43(9): 1116-25. Sympathetic skin responses following both electrical nerve and magnetic brain stimulations in insulin-dependent diabetic patients show an early yet detectable impairment of afferent pathways that takes place before the onset of peripheral neuropathy or dysautonomia, as is disclosed in a 1999 article by L. Sagliocco, F. Sartucci, O. Giampietro, and L. Murri entitled “Amplitude loss of electrically and magnetically evoked sympathetic skin responses in early stages of type 1 (insulin-dependent) diabetes mellitus without signs of dysautonomia” published in Clinical Autonomic Research: Official Journal of the Clinical Autonomic Research Society 9(1):5-10). The conduction of vibrations from tuning forks is being used to screen for sensation loss that can expose the diabetic patient to the risk of foot injury, as is disclosed in the 1990 article by P. H. Tchen, H. C. Chiu, and C. C. Fu entitled “Vibratory perception threshold in diabetic neuropathy” published in Journal of the Formosan Medical Association 89(1):23-9 and in the 1990 article by C. Liniger, A. Albeanu, D. Bloise, and J. P. Assal J P entitled “The tuning fork revisited” published in Diabetic Medicine 7(10):859-64. Functional changes in pulsatile arterial blood flow occur early in the time course of insulin-dependent diabetes and can be detected by measuring pulsatile waveforms noninvasively using an electromagnetic flowmeter, as is disclosed in a 1983 article by L. N. Cunningham, C. Labrie, J. S. Soeldner, and R. E. Gleason entitled “Resting and exercise hyperemic pulsatile arterial blood flow in insulin-dependent diabetic subjects” published in Diabetes 32(7):664-9. A non-invasive evaluation of lens fluorescence has been suggested as an early indicator of ocular complications associated with diabetes as is disclosed by M. Mota, A. M. Morgado, A. Matos, P. Pereira, and H. Burrows in 1999 in their article entitled “Evaluation of a non-invasive fluorescence technique as a marker for diabetic lenses in vivo” published in Graefe's Archive for Clinical and Experimental Ophthalmology 237(3):187-192.

The prior art devices and processes discussed above generally are not suitable for automatically detecting and treating a multitude of chronic disease states, are not readily adapted to treat small, localized internal regions of a living organism, and cannot readily and automatically modify the treatment regimen as the condition of the living organism changes.

### SUMMARY OF THE INVENTION

In accordance with this invention, there is provided an implantable device comprised of means for emitting and delivering energy to specific sites within a body, a programmable controller for varying the type and/or amount of energy emitted, and means for sensing a condition of a biological organism. The energy emitted by the device comprises part or all of the spectra of a desired energy pattern, and it contains at least a major peak and a minor peak.

### BRIEF DESCRIPTION OF THE DRAWINGS

The invention will be described by reference to the following drawings, in which like numerals refer to like elements, and in which:

FIG. 1 is a schematic of one preferred implantable device of this invention disposed within a patient;

FIG. 2 is a block diagram of one possible process for determination and subsequent utilization of an energy pattern;

FIGS. 3 through 8 are schematic diagrams of various arrangements of one or more implantable devices disposed within a patient;

FIG. 9 is a flow diagram of the operation of the device of FIG. 4;

FIGS. 10, 11, and 12 are graphs of some of the energy patterns delivered to a patient in one of the preferred processes of this invention;

FIG. 13 shows a shunt configuration;

FIG. 14 is a schematic showing utilization of the invention in a tube or pipe;

FIG. 15 is a schematic showing utilization of the invention in a fluid holding vessel;

FIG. 16 is a schematic diagram of three types of energy emitting devices in accordance with embodiments of the invention;

FIG. 17 is a schematic diagram of another energy emitting device in accordance with embodiments of the invention;

FIG. 18 is a block diagram of process for treatment of diseased cells;

FIG. 19 is a schematic diagram of a stent with a light emitting coating in accordance with embodiments of the invention;

FIG. 20 is a schematic diagram of two devices for treatment of congestive heart failure; and

FIG. 21 is a schematic illustration of a device for interrogating cellular components.

### DESCRIPTION OF THE PREFERRED EMBODIMENTS

In one embodiment of this invention, spectral analyses is used to describe some of the properties of a specified electromagnetic energy pattern. The term spectral analysis, as used in this specification, refers to the determination of the distribution of frequencies or wavelengths of transmission or absorption, or both, within the energy spectrum; it is an analytical technique for identification of materials, or of electromagnetic, vibrational, rotational frequencies. See, e.g., U.S. Pat. No. 6,191,417 (mass spectrometer), U.S. Pat. Nos. 6,191,271, 6,043,276, 5,902, 772, 5,814,314, 5,565,037, 5,462,751, 5,334,394, 4,997,842, and the like. The entire disclosure of each of these United States patents is hereby incorporated by reference into this specification.

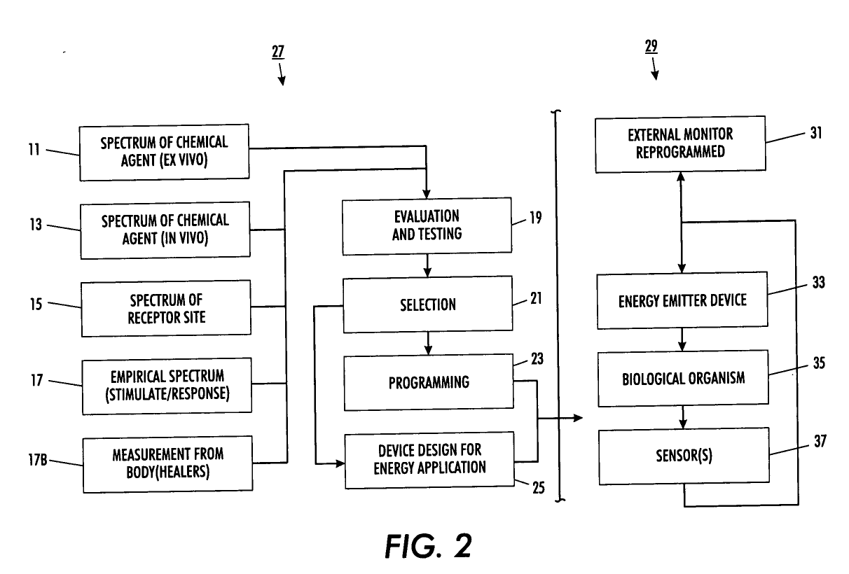

Similarly, the terms energy spectrum or spectrum or spectra or energy pattern, are used in this specification. These terms refer to the set of electromagnetic, vibrational, rotational, or other energy type, pattern of frequencies. Frequencies and waveforms can be combined in different ways including, but limited to, amplitude modulation, frequency modulation, pulsating direct current, square wave, sawtooth waves, ramping, etc.) These patterns may be determined or composed by means illustrated in FIG. 2.

One embodiment of this invention involves the application of an electromagnetic energy to an organism. The organism used in the process of this invention may be, but need not be, a living biological organism. Thus, by way of illustration and not limitation, the processes of this invention may be used with organs harvested from people who recently have died and wish to donate such organs.

The organism may be an animal organism, such as, e.g., a human being, a mammal, a reptile, and the like. Alternatively, or additionally, the organism may be a vegetable organism, such as a food crop. Alternatively, or additionally, the organism may be a virus, a bacteria, a mold, a yeast, a protozoa, and/or one or more other life forms.

By way of further illustration, one may treat via the process of this invention genetically modified bacteria used in cell cultures. In one aspect of this embodiment, the organisms used in fermentation processes (such as, e.g., making bread, brewing alcohol) may be treated with one or more forms of energy to insure their viability and/or optimal performance.

By way of further illustration, one may use one or more radiations in hydroponic farming to increase the yield of certain crops.

By way of further illustration, one may implant an energy emitting device into a tree and/or plant to increase its growth and/or production and/or disease resistance.

It is known that very minute alterations to molecules and fluids, such as blood or water, can have dramatic therapeutic effects, and that it is possible to digitize the method for effecting the alterations of these treatments and transmit them electronically so that they can be repeated with high precision at a later time and if necessary in a different place. As a result, complex diagnostics, including imaging and chemical analyses, can be conducted of tissue or fluid samples at a remote site, and a patient prescription provided for treating the situation that can be transmitted to the patient location and administered locally.

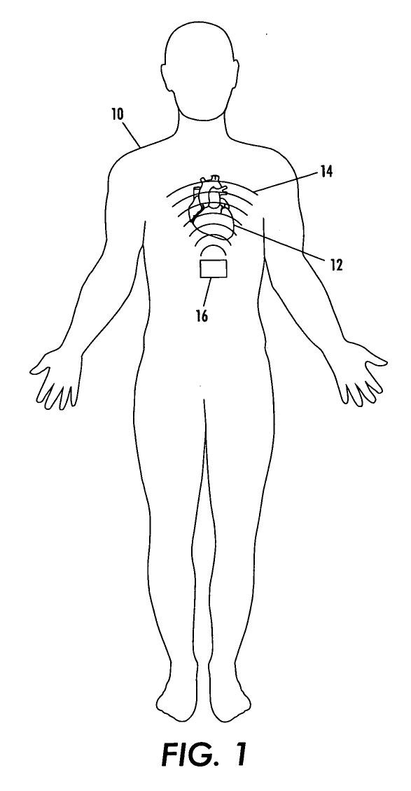

FIG. 1 is a schematic of one implantable device of this invention. Referring to FIG. 1, and in the preferred embodiment depicted therein, it will be seen that an energy emitter 16 is implanted into a biological organism 10, preferably in the proximity of an organ 12. In the embodiment depicted, the emitter 16 emits photonic or other electromagnetic energy 14 onto organ 12. The energy 14 may, e.g., be electrostatic, magnetostatic, acoustic, or very low frequency (VLF) through ultraviolet electromagnetic signals.

In one embodiment, the emitter 16 is utilized to effect a process for treating the body 10. In this process, one first determines the electromagnetic pattern of a biological process within body 10. This energy pattern determination may be made, e.g., by the process depicted in FIG. 2. Once the electromagnetic or other energy pattern has been determined, a portion of said energy pattern may be directly applied within the body 10. The energy pattern preferably is characterized by at least one major peak and one minor peak in its spectrum.

In one preferred embodiment, the energy emitted by emitter 16 varies with time in either its frequencies and/or amplitudes and/or phases. In another preferred embodiment, the energy spectrum emitted by emitter 16 varies with time. Thus, by way of illustration, one may transmit the spectra of a drug as it dissolves in the organism and interacts with the organism over time.

In another embodiment, the energy emitted by emitter 16 has a spectrum with at least one major peak and one minor peak. In another embodiment, the energy emitted by emitter 16 contains at least 5 major peaks and minor peaks.

In one embodiment, the energy emitted by emitter 16 has at least 10 major and/or minor peaks.

In one embodiment, the energy emitted by emitter 16 is a combination of energies selected from the group consisting of photonic energy, vibratory energy, electrical energy, and mixtures thereof, provided that, in this embodiment, at least two of such energies are emitted.

In one aspect of this embodiment, millimeter and/or centimeter wavelength energy is used. In general, this energy has a frequency of from about 30 to about 300 gigahertz. In some papers, reference to “millimeter waves” refers to frequencies around 60 gigahertz.

By way of further illustration, one may use energy of from about 1 to about 3 hertz to regenerate nerves. One may use an energy of from about 5 to about 9 hertz to promote bone growth. One may use an energy of about 10 hertz to heal ligaments. Energies of 15, 20, and 72 hertz decrease skin necrosis, stimulate capillary formation, and cause the proliferation of fibroblasts. Energies of 25 and 50 hertz promote synergistic effects with nerve growth factor. In general, the use of energies from about 1 to about 100 hertz promotes healing of many bodily parts.

Resistant myofascial pain can be treated with microcurrent of specific frequencies, as is disclosed in a 1998 article by C. McMakin entitled “Microcurrent treatment of myofascial pain in the head, neck, and face” published in Topics in Clinical Chiropractic 5(1):29-35. Chronic wounds can be treated by electric and electromagnetic fields, as is disclosed in a 1992 article by L. Vodovink and R. Karba entitled “Treatment of chronic wounds by means of electric and electromagnetic fields. Part 1. Literature review” published in Medical and Biological Engineering &_Computing 30:257-266. A variety of soft tissues have been treated with pulsing electromagnetic fields and 27 megahertz electromagnetic frequencies, as is disclosed by B. F. Sisken and J. Walker in an article published in 1995 with the title “Therapeutic aspects of electromagnetic fields for soft-tissue healing” in Advances in Chemistry Series 250, American Chemical Society, Washington D.C., pp. 277-285. Photoradiation therapy has been used for the treatment of malignant tumors, as was disclosed in 1978 by T. J. Dougherty, J. E. Kaufman, A. Goldfarb, K. R. Weishaupt, D. Boyd, and A. Mittleman A in an article entitled “Photoradiation therapy for the treatment of malignant tumors” published in Cancer Research 38:2628-2635). Weak direct current fields or stronger alternating current fields enhance the sprouting of intact saphenous nerves in rats, as is disclosed in an article by B. Pomeranz, M. Mullen, and H. Markus in 1984 with the title “Effect of applied electrical fields on sprouting of intact saphenous nerve in adult rat” published in Brain Research 303:331-336; and electrical fields enhance the regeneration of spinal cord in the lamprey, as is disclosed by R. B. Borgens, E. Roederer and M. J. Cohen in a 1981 article entitled “Enhanced spinal cord regeneration in lamprey by applied electric fields” published in Science 213:611-617. Scalar waves have been used to stimulate the immune system, as is disclosed by G. Rein in a 1989 article entitled “Effect of non-hertzian scalar waves on the immune system” published in the US Psychotronic Association Journal 1:15, and in another article by G. Rein published in 1998 entitled “Biological Effects of Quantum Fields and their Role in the Natural Healing Process” published in Frontier Perspectives 7(1):16-23. Skin wounds and intractable ulcers have been stimulated to heal faster with application of electrical fields, as is disclosed by D. S. Weiss, R. Kirsner, and W. H. Eaglstein in 1990 in an article entitled “Electrical stimulation and wound healing” published in Archives of Dermatology 126:222-225. Infrasound has been used in a wide variety of clinical situations, as is disclosed by R. R. Sunderlage in 1996 in a paper entitled “Clinical applications of infrasound therapy and clinical case studies” published as a research paper submitted to the Midwest Center for the Study of Oriental Medicine, course #A572, Dec. 21, 1996. Low frequency current pulses have been used over many years in electroacupuncture, as is disclosed by R. Voll, et. al. and summarized in the 1999 book Virtual Medicine by K Scott-Mumby and published by Harper Collins, London. Externally applied picotesla magnetic fields have been used to treat neurologic disorders as disclosed by J. I. Jacobson and W. S. Yamanashi in 1994 in an article entitled “A possible physical mechanism in the treatment of neurologic disorders with externally applied picotesla magnetic fields” published in Subtle Energies 5(3):239-252.

Laser acupuncture has been used to treat paralysis in stroke patients, as is disclosed by M. A. Naeser, M. P. Alexander, D. Stiassny-Eder, V. Galler, J. Hobbs, D. Bachman, and L. N. Lannin in 1995 in an article entitled “Laser Acupuncture in the Treatment of Paralysis in Stroke Patients: A CT Scan Lesion Site Study” published in the American Journal of Acupuncture 23(1):13-28. In general, the use of energies from about 1 to about 100 hertz promotes healing of many bodily parts, with some studies showing effects at much higher frequencies into the terahertz range. Millimeter waves are being utilized for the treatment of pain as disclosed by A. A. Radzievsky, M. A. Rojavin, A. Cowan, S. I. Alekseev, A. A. Radzievsky Jr, and M. C. Ziskin in a 2001 article entitle “Peripheral neural system involvement in hypoalgesic effect of electromagnetic millimeter waves” published in Life Science 68(10): 1143-51.

U.S. Pat. No. 4,528,256 discloses that cells can be modified “by subjecting them to the influence of ions from a metal electrode, for example of silver, which is placed in contact with them and which is made electrically positive, causing low intensity direct current to flow through them. The cells, which are relatively specialized, such as normal mammalian fibroblasts, assume a simpler, relatively unspecialized form and come to resemble hematopoetic or marrow-like cells.” The process leads to improved therapeutic effects, “such as enhanced cell or biochemical production, enhanced lesion healing, enhanced normal tissue growth or regeneration, cell dedifferentiation, changing cancer cell form, and stopping multiplication of cancer cells.” The entire disclosure of this patent is hereby incorporated by reference into this specification.

A light source called the MFbio-spectrum lamp treatment has been successful in treatment of diabetes, as is disclosed by G. Wu in 2000 in an article published on the web at http://www.findhealr.com/mall/telstar/clinic/diabetes.php3). Pulsed electromagnetic fields are being used to stimulate cutaneous wound healing in diabetic rats, as is disclosed by O. Patino, D. Grana, A. Bolgiani, G. Prezzavento, J. Mino, A. Merlo, and F. Benaim in a 1996 article entitled “Pulsed electromagnetic fields in experimental cutaneous wound healing in rats” published in the Journal of Burn Care Rehabilitation 17(6 Pt 1):528-31. Magnetotherapy is being applied to the comprehensive treatment of vascular complications of diabetes mellitus, as is disclosed by I. B. Kirillov, Z. V. Suchkova, A. V. Lastushkin, A. A. Sigaev, and T. I. Nekhaeva in a 1996 article entitled “Magnetotherapy in the comprehensive treatment of vascular complications of diabetes mellitus” published in Klinicheskaia Meditsina (Moskva) 74(5):39-41). Pulsating high-frequency electromagnetic fields are being used to treat patients with diabetic neuropathies and angiopathies, as is disclosed by V. Vesovic-Potic and S. Conic in a 1993 article entitled “Use of pulsating high-frequency electromagnetic fields in patients with diabetic neuropathies and angiopathies” published in Srpski Arhiv Za Celokupno Lekarstvo (Beograd) 121(8-12):124-6. Suppurative wounds in patients with diabetes mellitus are being treated by magnetic field and laser irradiation, as is disclosed by R. A. Kuliev, R. F. Babaev, L. M. Akhmedova, and A. I. Ragimova in a 1992 article entitled “Treatment of suppurative wounds in patients with diabetes mellitus by magnetic field and laser irradiation” published in Khirurgiia (Moskva) (7-8):30-3). Electromagnetic stimulation of the rat pancreas lowers serum glucose levels in rats, as is disclosed by P. O. Milch, J. B. Ott, R. J. Kurtz, and E. Findl in a 1981 article entitled “Electromagnetic stimulation of the rat pancreas and the lowering of serum glucose levels” published in Transactions—American Society for Artificial Internal Organs 27:246-9). Non-invasive electromagnetic flowmetry (NMF) using external magnets and flowmetry by NMR are being used for screening for arterial diseases, monitoring of the treatment, and study of hardened arteries in diabetes, as is disclosed by H. Boccalon in 1989 in an article entitled “The necessary advantage of measuring the pulsatile arterial flow of the limbs in patients with arterial disease” published in Annales de Cardiologie et d Angeiologie (Paris) 38(8):461-4).

Referring again to the Figures, and in one embodiment, the energy utilized in the process of this invention has a frequency of at least 1,000 gigahertz (one terahertz) and is believed to cause deoxyribonucleic acid to resonate. In this embodiment, a multiplicity of different frequencies, each of which has a frequency of at least one terahertz, are used.

FIG. 2 is a flow diagram which illustrates one preferred embodiment of the energy pattern determination process of this invention. In step 11 of the process, the spectrum of a therapeutic agent is determined by spectral analysis, or by reference to standard tables of the spectrum of the agent.

In one embodiment, one may determine the vibrational spectrum of the agent by conventional means. Thus, e.g., one may determine the vibrational spectrum of a drug by the means disclosed in one or more of U.S. Pat. Nos. 6,232,499, 6,040,191, 5,912,179, 5,866,430, 5,848,977, 5,733,739, 5,733,507, 5,712,165, 5,555,366, 5,386,507, and the like. The entire disclosure of each of these United States patents is hereby incorporated by reference into this specification. Reference also may be had, e.g., to John A. Dean's “Analytical Chemistry Handbook”(McGraw-Hill, Inc., New York, 1995).

In another embodiment, one may determine the electromagnetic spectrum of the therapeutic agent; see, e.g., U.S. Pat. Nos. 6,178,346 and 5,210,590 (rapid scanning spectrographic analyzer), and the like, the entire disclosure of each of which is hereby incorporated by reference into this specification. Thus, e.g., one may determine the optical spectrum of the therapeutic agent by the means disclosed in United States patents, U.S. Pat. Nos. 6,251,280, 6,246,901, 6,167,297, 5,977,162, 5,853,370, 5,833,603, 5,622,945, 5,410,045, 5,330,741, 5,135,717, 4,980,566, 4,711,245, 4,250,394, and the like, the entire disclosure of each of which is hereby incorporated by reference into this specification.

Referring again to FIG. 2, and in the preferred embodiment depicted therein, in step 11 the spectrum of a chemical agent is determined ex vivo, outside of a biological organism. Alternatively, or additionally, one may determine the spectrum of a chemical agent in vivo in step 13 by conventional means. In both step 11 and 13, one may determine the spectrum of only one agent, or of two or more agents, in various combinations and at various concentrations. Alternatively, or additionally, one may determine the spectrum of one or more agents over a period of time. As is known to those skilled in the art, a drug within a biological organism will change its physical and/or chemical identity, due to dissolution in one or more solvents and/or reaction with one or more agents within the body. As the physical and chemical properties of the drug change, so does its spectrum.

It is known that signal molecules can activate their corresponding receptor sites without physical contact. See, e.g., an article by C. W. Smith, “Electromagnetic effects in humans,” in Biological Coherence and Response to External Stimuli, Frohlich H (editor), Springer-Verlag, Berlin, pages 205-232. Reference also may be had to James L. Oschman's book Energy Medicine: The Scientific Basis (Churchill Livingston, New York, N.Y., 2000) and a book published in 1957 by A. Szent-Gyorgyi entitled Bioenergetics, published by Academic Press, New York. In one preferred process of this invention, the energy patterns from signal molecules are used without their corresponding drugs to treat the receptor sites. Once one has determined a desired receptor response produced by a specified drug or combination of drugs, one may then evaluate which combination of energy pattern stimuli will produce the same response in step 19 of the process. Reference may be had to U.S. Pat. No. 6,242,209. The entire disclosure of this United States patent is hereby incorporated by reference into this specification.

Alternatively, and as is illustrated in step 15, one may determine the spectrum response of a receptor site to various stimuli, including stimulation by drugs as well as stimulation by application of various energy patterns or by combinations. Alternatively, one may determine the spectrum of the receptor site, over time, as it is exposed to a drug. By trial and error, one may determine what combination of stimuli produce the desired receptor response.

The set of drug compounds available to the medical community is limited by available chemical synthesis technology, and by precursor chemical structures available from organic, inorganic, botanical, or animal sources. Thus there is a practical limit to the array of signal molecules that can be used to elicit a cellular response. The cells themselves, and the receptor sites in particular, are under no such restriction. Thus there are a wide variety of electromagnetic spectra that have no corresponding available synthesized chemical structure, but which spectra may have effective, and even superior, therapeutic affect at the desired receptor site when used in the manner described in this invention. One approach to determine the specific receptor spectrum is to excite the receptor with an ultra short energy pulse, measure the resulting spectrum, and perform a mathematical transform on the resulting spectrum to determine the ideal-fit complementary spectrum that would be associated with the ideal-fit chemical compound. This approach shall be referred to as ‘receptor response spectrum development’ for descriptive purposes and is included in FIG. 2, step 15.

Alternatively, electromagnetic signals can be designed on the basis of highly specific information on the structure and operation of receptor sites on and within cells. The growing information on the molecular configurations of receptors and on the mechanisms taking place when ligands interact with receptors provides a wealth of opportunities for the design of highly specific electromagnetic therapies. We now know that what has been referred to in the past as “a receptor” can actually have several functional domains. There is a ligand-binding domain and an effector domain. The ligand-binding domain is the specific site, often on the cell surface, where a regulator ligand (such as a hormone, growth factor, or neurotransmitter) has its primary action. The effector domain consists of a series of intermediary cellular molecules in the signal transduction pathway. Drugs and electromagnetic fields can interact with both of these domains, including the domains of the second messenger molecules that convey messages within cells. Recent advances in computational chemistry, structural analysis of organic compounds, and biochemical measurement of the primary actions of drugs at their receptors have permitted the design of new and more specific drugs. The same information can be used in the de novo design of highly specific electromagnetic interventions. Moreover, recent advances in determining the structures of drug-receptor complexes, at atomic resolution by X-ray crystallography or nuclear magnetic resonance spectroscopy, are even more helpful, and offer great promise for the design of electromagnetic signals of extreme potency and specificity.

Alternatively, as is illustrated in FIG. 2, step 17, one may determine a therapeutic energy pattern by subjecting the body and/or individual organs, tissues, bodily fluids, cells, cells in culture to various energy patterns and recording the response.

Alternatively, as illustrated in FIG. 2, step 17B, one may determine a therapeutic energy pattern by measuring the energy patterns of a healthy body, and/or individual organs, tissues, bodily fluids, cells. Additionally, the energy patterns emitted by the body as one is placed into various meditative states may be recorded. Thus, e.g., the hands, e.g., can emit a range of electromagnetic frequencies from about 0.3 hertz to 30 hertz (see, e.g., an article by J. Zimmerman, 1985 “New technologies detect effects of healing hands” published in Brain Mind Bulletin 10 (September 30 issue, p. 3)). As disclosed by Zimmerman, the emitted electromagnetic energy may sweep through this frequency range rather than being a fixed frequency.

Referring again to FIG. 2, once a desired energy spectrum, or portion thereof, or combination of one or more such spectra, is identified, it may be evaluated in step 19 against other candidate spectra. The response of a biological body, or a portion thereof, can be determined, and a correlation can be made between the use of a specified spectrum and/or spectra and the response of the organism. Thereafter, in step 21 of the process, a spectrum and/or spectra may be selected for any particular condition to be treated in the biological organism; and information about this selected spectrum/spectra may be incorporated into a program in step 23. In step 25, the program may be incorporated into a device which is capable of sensing the condition within the biological organism, selecting the appropriate spectrum/spectra from its database, emitting such energy pattern and directing it to the appropriate site within the organism, sensing the response of the living organism to such emission, modifying such emission as appropriate, and/or ceasing such emission as appropriate.

In portion 27 of the process, which is comprised of steps 11 through 25, the steps necessary to identify the appropriate energy pattern are described. In portion 29 of the process, comprising steps 31 through 37, the steps necessary to apply the selected energy pattern to the living organism are described.

In step 31 of the process, which is optional, one may utilize an external monitor/reprogrammer for bidirectional communication between the implanted device and the outside world. With such a monitor/reprogrammer, one can visually observe indicia of the state of biological organism and, as appropriate, change the program of the implanted device.

The external monitor/reprogrammer is operatively connected to the implanted energy device of step 33 which, in response to external stimuli and/or in vivo stimuli provided by the biological organism, provides energy to biological organism of step 35. In one embodiment, depicted in step 37, a sensor which can monitor the response of the living organism to the applied energy and, with use of a programmable computer (not shown), continually modifies the energy delivered to the organism. The connection between the external monitor/reprogrammer 31 and the energy device may be direct, or it may be indirect. In one embodiment, the connection is indirect and is made, e.g., by means of transceivers.

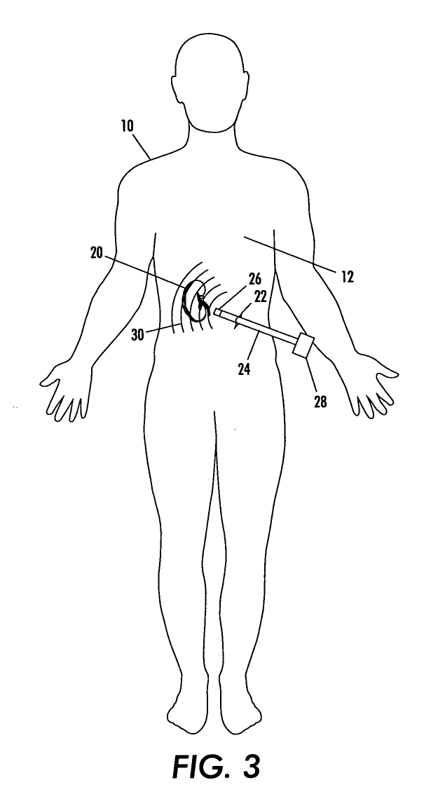

In another embodiment of this invention, illustrated in FIG. 3, an emitter 26 is attached to the end of a catheter 24 and is controlled by a controller 28. In the embodiment depicted in this FIG. 3, the catheter is inserted into body 10 through an incision 22. The organ 20 is then irradiated with the electromagnetic energy 30. An operator, not shown, may control the electromagnetic energy by adjusting parameters of the controller 28.

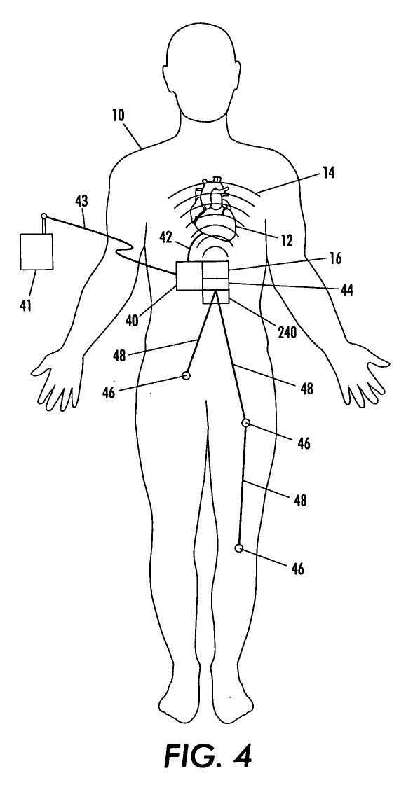

In another embodiment of this invention, illustrated in FIG. 4, an emitter 16 is an augmentation module connected to an implanted heart pacemaker 40; in the embodiment, the pacemaker 40 is connected via lead 42 to the heart 12. This augmentation module may be attached to the pacemaker 40 at any future date after the pacemaker 40 has been implanted without removal or otherwise replacement of the original pacemaker 40. Alternatively, the augmentation module may be implanted at the same time as the pacemaker 40. In either situation, the augmentation unit may be detached from the pacemaker 40 and removed from the body 10 at any time without significant disruption of the pacemaker 40. A controller with a programmable logic unit 44 is connecter to the emitter 16 and the pacemaker 40. The controller 44 also has communication means 48 to implanted sensors 46. The emitter 16 may be activated by the analyses of the sensors' input and comparison to threshold conditions or comparison to a programmable database of deleterious conditions. The emitted energy 14 may be adjusted from very low frequency to ultraviolet or terahertz range frequency programmatically through the controller's programmable logic unit 44. The emitted electromagnetic or vibrational energy signals produced by the augmentation modules may be a reproduction of the natural energy signals emitter from a healthy organ. In this way, a healthy signal may reinforce a non-healthy organ as well as to propagate a healthy signal to other organs.

Referring to FIG. 4, the sensors 46 are capable of determining the electromagnetic pattern and/or other physiological and/or biochemical and/or biophysical parameter of any portion of the body 10 while such body is functioning. One may determine the electromagnetic pattern of such body when, e.g., the liver is functioning properly. One may determine the electromagnetic pattern of such body when, e.g., the liver is not functioning properly. One may, e.g., determine the electromagnetic pattern of the heart in relation to diagnostic indicators of susceptibility to arrhythmias of various kinds. One may, e.g., determine the electromagnetic pattern of such body when the liver is exposed to one or more drugs, or to heat, or to any treatment. By making these measurements, one can correlate the optimum performance of, e.g., the liver with optimum electromagnetic patterns. Similar correlations can be made with other organs and/or bodily processes.

Once such correlations have been made, using the methods disclosed herein or by reference to research studies conducted by others, one can deliver to the patient, via emitter 16, that portion of the spectral pattern which is advantageous to the patient at times when it is advantageous to the patient. Thus, e.g., the sensors 46 can determine when, e.g., the liver is malfunctioning and deliver the required electromagnetic radiation to the patient, either alone and/or in combination with one or more drugs, until the liver is functioning properly.

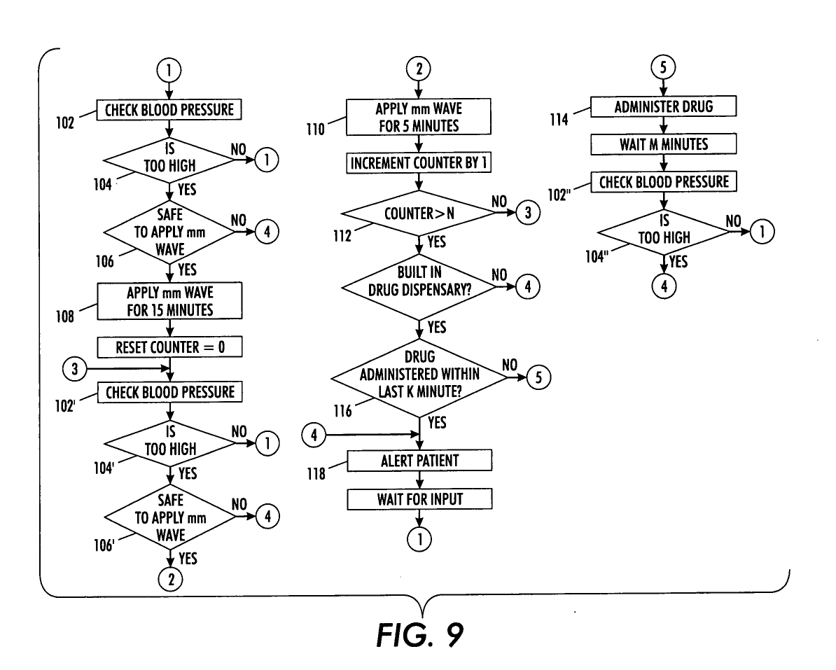

In the preferred embodiment depicted in FIG. 4, an implantable drug dispenser 240 is operatively connected to the controller 44 and, as required, delivers one or more drugs in response to the commands of such controller 44. As will be apparent to those skilled in the art, the process depicted in FIG. 9 may be effected by the device depicted in FIG. 4.

In one embodiment, as depicted in FIG. 4, the sensors 46 are so constructed and situated as to detect energy patterns in the environment, external to the body 10. Controller 44 can analyze such patterns and can determine if such external energy patterns are disruptive to the body 10 or to the treatment currently administered. If such a disruptive external energy pattern is detected, the controller 44 may change the energy pattern emitted from emitter 16 or halt the administration of treatment until the disruptive external energy patterns are no longer detected and/or notify the patient through communications device 41 using communication channel 43. Communications channel 43 may be, e.g. by radio frequency means.

In another embodiment of this invention, also depicted in FIG. 4, the sensors 46 are so constructed and situated as to detect disturbances in the interplanetary and/or geomagnetic fields that have been correlated with vulnerability to myocardial infarction, cardiac arrhythmias, stroke, seizures, depression, and mortality in general, as is disclosed by Y. I. Gurfinkel, V. V. Lyubimov, V. N. Orayevskii, L. M. Parfenova and A. S. Yur'ef in a 1995 article entitled “Effect of Geomagnetic disturbances on Capillary Blood Flow in Patients Suffering from Ischaemic Disease of the Heart” published in Biophysics 40(4):777-783′ in a 2001 article by Y. I. Gurfinkel, V. L. Voeikof, E. V. Buravlyova and S. E. Kondakov entitled “Effect of Geomagnetic Storms on the Erythrocyte Sedimentation Rate in Ischaemic Patients” in Critical Reviews in Biomedical Engineering published by Begell House, Inc; in a 1995 article by G. Villoresi, T. K. Breus, L. I. Dorman, N. Iucci and S. I. Rapoport entitle “Effect of Interplanetary and Geomagnetic Disturbances on the Rise in the Number of Clinically Severe Medical Pathologies (Myocardial Infarction and Stroke)” published in Biophysics 40(5):983-993; in a 1995 article by F. J. Lucatelli and E. J. Pane entitled “Correlation Between Cosmophysical Factors and the Onset of Manic-Depressive Psychosis” published in Biophysics 40(5):1023-1027; in a 1998 article by T. L. Gulyaeva entitled “Mortality Correlates of Cosmic and Meteorological Factors” published in Biophysics 43(5):789-795; in a 1976 article by K. Venkataraman with the title “Epilepsy and Solar Activity—An Hypothesis” published in Neurology India 24:148-152; and in a 1981 article by M. Rajaram and S. Mitra entitled “Correlation Between Convulsive Seizure and Geomagnetic Activity” published in Neurosciences Letters 24:187-191. Other disorders have been correlated with very low frequency atmospherics or VLF-sferics caused by atmospheric discharges (lightening), as is disclosed by A. Schienle, R. Stark, and D. Vaitl in a 1998 article with the title “Biological Effects of Very Low Frequency (VLF) Atmospherics in Humans: A Review” published in the Journal of Scientific Exploration 12(3):455-469. Controller 44 can analyze such environmental patterns and can determine if such patterns are disruptive to the body 10 or to the treatment currently administered. If such a disruptive external energy pattern is detected, the controller 44 may change the energy pattern emitted from emitter 16, halt the administration of treatment until the disruptive external energy patterns are no longer detected, introduce a protective energy pattern such as an enhanced pacemaker signal, and/or notify the patient through communications device 41 using communication channel 43.

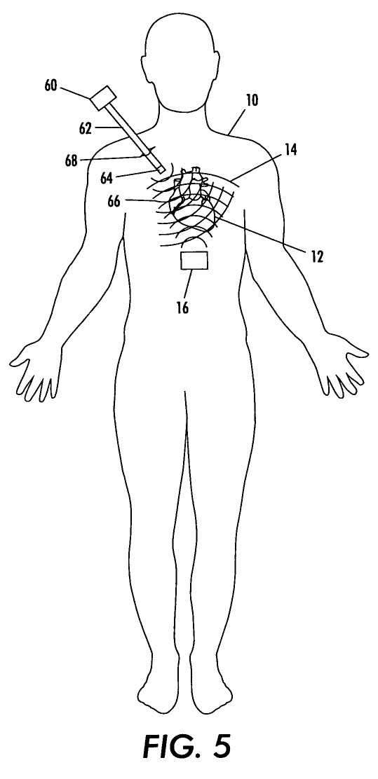

In another embodiment of this invention, illustrated in FIG. 5, an emitter 16 implanted in body 10 emits electromagnetic energy 14 onto or within an organ 12. Additionally, a probe 62 with an emitter 64 at the insertion tip of a catheter is inserted into the body 10 through incision 68. The inserted probe emitter 64 emits electromagnetic energy 66 onto the organ 12 from a different orientation than that of emitter 16. The electromagnetic energy 66 which is emitted from the probe emitter is controlled via controller 60 and need not have the same characteristics as the electromagnetic energy emitted from emitter 16.

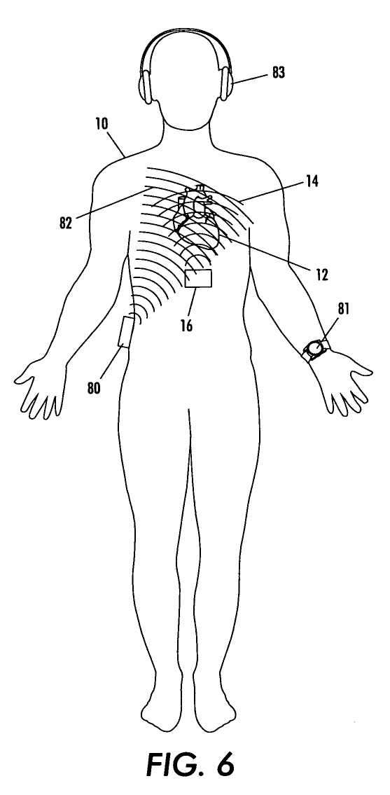

FIG. 6 illustrates an embodiment in which an emitter 16 implanted in body 10 emits electromagnetic energy 14 onto an organ 12, and an external device 80 delivers vibratory energy 82 to the organ 12. In one aspect of this embodiment, emitter 16 continually emits energy, whereas external device 80 intermittently emits energy. Other external devices 81 and 83 may also deliver various energy patterns to the body 10.

In one embodiment, illustrated in FIG. 6, electromagnetic energy may be delivered through a device located outside of the patient's body, such as a watch 81 and/or external appliances 80 and/or 83 and/or in glasses frames (not shown) ankle bracelets (not shown), etc.

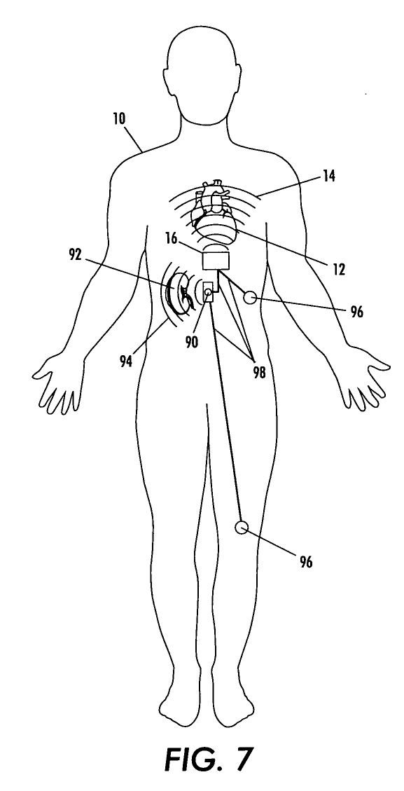

In another embodiment of this invention, illustrated in FIG. 7, an electromagnetic emitter 16 implanted in body 10 emits electromagnetic energy 14 onto an organ 12. Additionally, a vibrational energy emitter 90 is also implanted into body 10 and delivers vibrational energy 94 to organ 92. The emitter 90 also consists of a sensor element. Additionally, other sensor devices 96 are implanted into body 10. All of such implanted devices are in communication through communication channels 98 to form a network. The communication means may be through fiber optic cables, wires, shielded wires, wireless or other means. The implanted devices 16 and 90 are so constructed as to contain programmable logic units suitable for analyzing signals from other implanted devices and from sensors 96 and to initiate the adjustment of adjustable parameters of any implanted device in the network. The emitters are so designed as to allow for multiple frequencies and intensities to be emitted at the same time including a carrier and pulsed waves combined. Each of these implanted devices is so constructed as to allow the addition of other implanted or external devices or the removal of said devices from the network of devices without the interruption of other devices in the network. The interconnection of these devices may be made by conventional means. See, e.g., U.S. Pat. No. 5,454,837. The entire disclosure of this United States patent is hereby incorporated by reference into this specification.

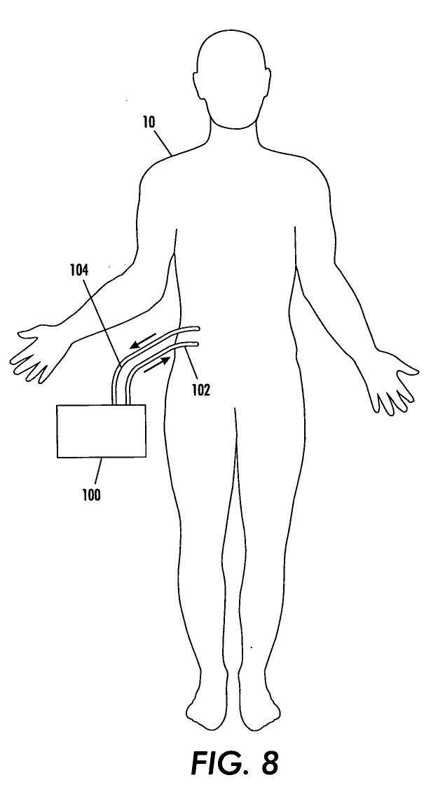

In the embodiment depicted in FIG. 8, bodily fluid is withdrawn from body 10 via line 104, treated with energy in device 100, and returned to the body via line 102. In this embodiment, a portion of the bodily fluid may be segregated in device 100 and treated separately from the other bodily fluid. Thus, e.g., the device 100 may comprise a flow cytometer which identifies cancerous cells, segregates them, treats them with high heat and/or radiation, and returns some or all of the cells so treated to the body.

In one preferred embodiment, in any or all of the processes of this invention, the electromagnetic energy is delivered directly into one or more bodily fluids, such as, e.g., the blood, the lymph, the urine, cerebrospinal fluid, endolymph, aqueous humor, etc. Reference may be had, e.g., to FIG. 8, in which a bodily fluid is treated in reservoir 100 after being removed from a body 10 and then returned to such body 10.

FIG. 9 is a flow diagram of one preferred process. In step 102 of the process, the emitter controller 16 (not shown) checks the blood pressure of the biological organism using, e.g., sensors 46 (see FIG. 4). If the blood pressure of the organism is lower than a specified level, in step 104 the process is aborted If the blood pressure of the organism is higher than such specified level, then in step 106 the controller (not shown) optionally checks other body parameters (such as, e.g., body temperature, pulse rate, etc.) to determine whether it is safe to apply to specified therapy.

After verifying that the therapy regimen is safe, in step 108, millimeter wave frequency is applied for a specified duration such as, e.g., 15 minutes. Thereafter, the blood pressure of the biological organism is again checked in step 102′. In one aspect of this embodiment, if the blood pressure of the organism is still too high after the initial treatment, additional incremental treatments 110 preferably are continued up to a threshold decision point 112. In the embodiment depicted, additional chemical therapy is administered in step 114, and monitored in step 102″. If this additional drug therapy is not effective, the patient is alerted in step 118.

It will be apparent to those skilled in the art that the preferred process depicted in FIG. 9 can have constructive application for a variety of other medical conditions besides the amelioration of high blood pressure. For example, another preferred embodiment is in the regulation of carbohydrate metabolism in the diabetic patient. Here the sensor in step 102 monitors the concentration of glucose in the blood and millimeter or other frequencies are emitted in step 108 to effect a stimulation of glucose absorption in the tissues in the body. Again, if the blood glucose concentration in the organism is still too high after the initial treatment, additional incremental treatments 110 preferably are continued up to a threshold decision point 112. In the embodiment depicted, additional electromagnetic energy is administered in step 108 or additional chemical therapy is administered in step 114, and monitored in step 102″. If this additional electromagnetic or drug therapy is not effective, the patient is alerted in step 118. The entire configuration, or suitable variations of it, constitute what has been termed an “artificial pancreas.”

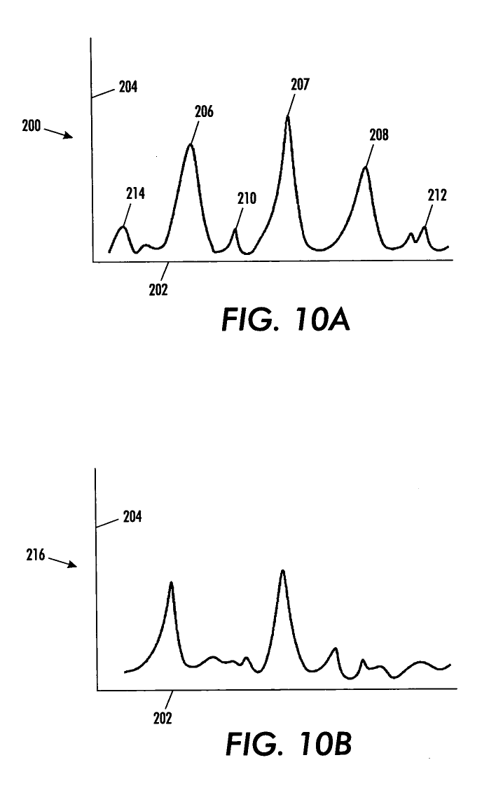

FIG. 10A is a graph of a spectrum 200 of one preferred energy pattern delivered from the emitter 16 to a patient 10 at “time zero.” In the graph of this FIG. 10A, frequency is plotted on the horizontal axis 202, and amplitude is plotted on the vertical axis 204.

Referring to the graph depicted in FIG. 10A, it will be seen that the spectrum 200 is comprised of major peaks 206, 207 and 208 and minor peaks 210, 212, and 214. In general, the spectrum of the energy emitted by emitter 16, in this embodiment, will contain at least two major peaks and two minor peaks.

The spectrum 216 depicted in the graph of FIG. 10B is illustrative of the pattern emitted by the same emitter 16 at some time, t1, after “time zero.” As will be apparent, in this embodiment, the spectrum 216 differs from the spectra 200.

When a drug is administered to patient, its spectrum changes as it is dissolved within the patient's system and/or is metabolized within the patient. As the drug undergoes physical and/or chemical changes, its spectrum changes. In one embodiment of this invention, the energy pattern delivered by the emitter 16 is substantially comparable to the energy pattern delivered by a drug as it undergoes physical and/or chemical changes within the patient's body.

One may, by conventional techniques, measure the spectrum of one or more drugs as they interact with and within a patient's body. Thereafter, one may program this spectrum into an emitter comprised of programmable computer such that the emitter will deliver the same energy pattern to a biological organism as the drug did, over time. Thus, e.g., one may use the emitter 16 and the controller 44, as depicted in FIG. 4.

It will be apparent to those skilled in the art that the process just described may not be ideal, as alterations in the structure of drug molecules, and resulting alterations in the emission spectrum of the molecules, may be detrimental to the organism, leading to undesired side effects. Hence in another preferred embodiment the computer is programmed such that the emitter will continue to deliver the same energy pattern to a biological organism as the drug did when the drug was first administered to the patient.

In the embodiment of FIG. 4, not only is both an emitter and controller present, but a multiplicity of sensors 46 also are present. Thus, with the apparatus depicted in FIG. 4, one may monitor the reaction of a patient's body to the administration of electromagnetic energy from the emitter and/or the administration of one or more drugs.

How the energy pattern of any particular drug, or combinations of drugs, or how combinations of drugs and electromagnetic fields, changes over time may be stored within the controller 44 of FIG. 4. The response of the patient's body to various portions of such energy patterns may be determined by the sensors 46 and the controller 44. In many cases, it will be determined that a certain portion of the spectral pattern, and/or its combination with one or more drugs, advantageously affects the patient's body. In other cases, it may be determined that a certain portion of the spectral pattern, and/or its combination with one or more drugs, disadvantageously affects the patient's body. The device of FIG. 4 will be capable of determining, at any particular point in time, which portion, if any, of the energy pattern and/or drug should be applied at that point in time. Thereafter, by monitoring the patient's reaction to the administered energy pattern(s) and/or drug(s), the controller 44 can cause the emitter 16 or the implantable drug dispenser 240 connected to the controller 44 to modify the energy pattern(s).

If, for example, a drug is being administered which, at a particular point in time, is producing a disadvantageous energy pattern, the emitter 16 may emit one or more interfering and/or phase shifted and/or phase inverted and/or complementary energy patterns which, after they interact with the energy pattern produced by such drug, or with the response of the receptor molecules the drug is acting upon, produce the desired energy pattern and/or lack thereof.

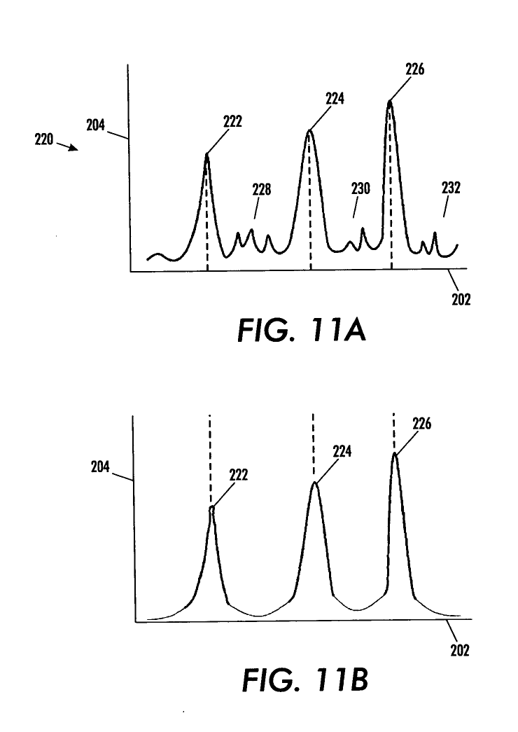

FIG. 11A illustrates a spectrum 220. In the particular embodiment depicted in FIG. 11A, and for a particular condition, it might be determined that only major peaks 222, 224, and 226 produce advantageous results but that the minor peaks in the “troughs” of the spectra, regions 228, 230, 232, have deleterious effects. In this case, as is illustrated in FIG. 11B, the controller 44 will cause emitter 16 to emit only the major peaks 222, 224, and 226. Alternatively, it may be determined that one or more of the major peaks is the cause of deleterious effects, in which case these major peaks are removed from the emitted spectrum.

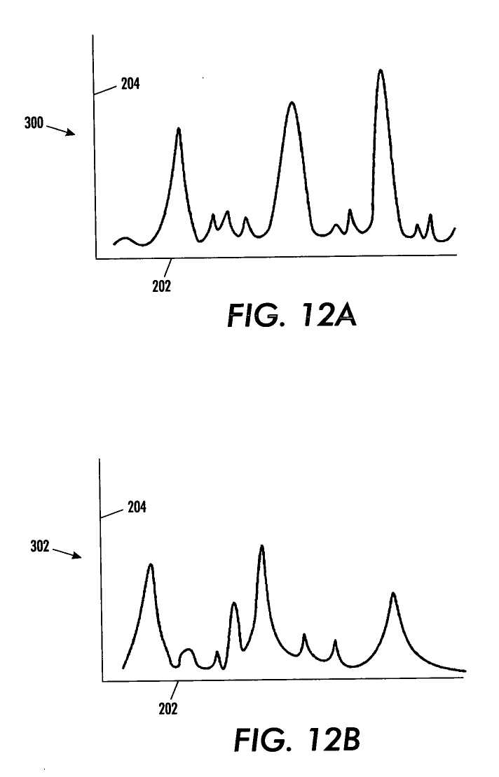

The process of this invention is not limited to the use of only one emitter 16 or only one implantable drug dispenser 240. As will be apparent to those skilled in the art, the use of a multiplicity of emitters 16 allows one to produce a large variety of different waveforms and spectra patterns that can interact with a multiplicity of injected drugs. FIGS. 12A and 12B illustrate the spectral patterns 300 and 302 which may be produced at one particular point in time by emitters 16 and 64 (see FIG. 5).

In one embodiment, there is provided an apparatus for treating a biological organism, comprising an externally worn and removable appliance comprised of means for inducing an electromagnetic and/or vibrational and/or light and/or other energy pattern of a biological process and/or a suitable drug or drugs through the skin of a organism which, preferably, is living. In this embodiment, the energy pattern corresponds to at least a portion of the electromagnetic pattern, or a modification thereof, of a biological process within the organism.

In many cases, it may be desirable to introduce more than one electromagnetic pattern to the patient. Thus, in one embodiment, depicted in FIGS. 7 and 12, there is provided a process comprising the steps of determining a first electromagnetic pattern of a biological process within a living organism, determining a second electromagnetic pattern of a biological process within a living organism, introducing said first electromagnetic pattern into said living organism, and introducing said second electromagnetic pattern into said living organism. As will be apparent, more than two such electromagnetic patterns may be administered, and they may be administered in combination with one or more drugs.

In one preferred embodiment, in any or all of the processes of this invention, the electromagnetic energy and/or other energy is delivered directly into cartilage. In another embodiment of the invention, the electromagnetic and/or other energy is delivered directly into bone. In yet another embodiment of the invention, the electromagnetic and/or other energy is delivered directly into brain cells. In yet another embodiment of this invention, the electromagnetic and/or other energy is delivered to fascia and/or cerebrospinal fluid and/or other fluids. In yet another embodiment of this invention, the electromagnetic and/or other energy is delivered to acupuncture and/or other biologically active points within and/or on the body.

In any or all of the aforementioned embodiments, one may substitute for part or all of the electromagnetic energy other energy forms, such as vibratory energy.

After a suitable number of correlations have been made with the devices of this invention, one may deliver one or more energy patterns, and/or drugs, adapted to provide anti-allergy signals, anti-aids recognition signals, signals that reduce the side effects of drugs, signals that mimic the signals of homeopathic remedies, signals that mimic the patterns of heat drugs (such as beta blockers), nitrolycerine, anti-tumor drugs, antibiotics, antiviral agents, stress reducing agents, pain killers, and the like. As will be apparent, this list is merely illustrative.

In one embodiment, a desired electromagnetic spectrum and/or modulated light or sound (including, e.g., ultraviolet light or ultrasound or infrared radiation, e.g.) is injected directly into a patient's blood stream on demand and/or at regular intervals and/or continuously.

In one embodiment, the spectral pattern which exists when the AIDS virus attaches to a lymphocyte is determined, and a pattern designed to interfere with this first spectral pattern is emitted. Thus, e.g., one may emit coherent photon signals that mediate the behavior of the AIDS viron and its attraction to and identification of and docking on the human lymphocyte. In one aspect of this embodiment, either the viron itself and/or a component of the viron is caused to resonate at its natural coherent resonant frequency. Two key elements of such viron are two surface proteins, glycoprotein GP41 and glycoprotein GP 120; they constitute a dielectric antenna. By the application of suitable electromagnetic energy to such “antenna,” the AIDS viron can be affected.

In one embodiment, the emitter 16 is comprised of means for transmitting a desired electromagnetic pattern to a pacemaker. Thus, e.g., one may transmit suitable analog, digital, or scalar versions of such signals to a cardiac assist device. In one aspect of this embodiment, the cardiac assist device is adapted to store the spectrum transmitted to it by the emitter 16 and, when appropriate, to retransmit part or all of such spectrum.

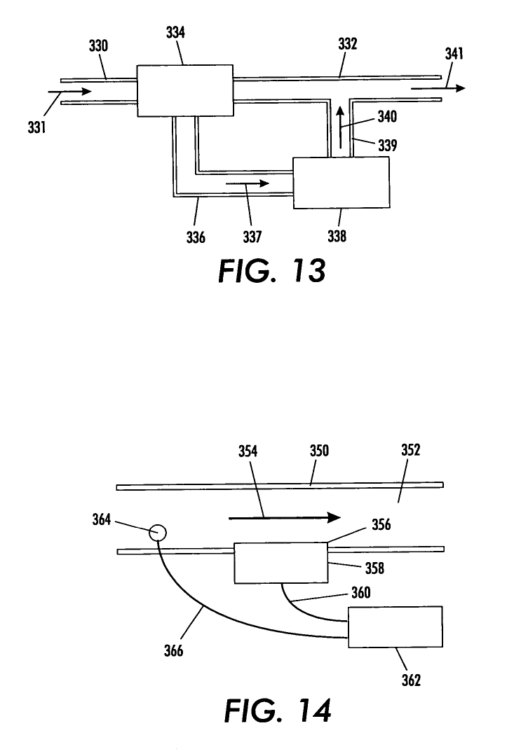

In another embodiment, see FIG. 13, an emitter is built into a shunt. Referring to FIG. 13, a blood artery or vein is divided into two parts 330, 332 and a filter/separator 334 is inserted between them. Arrows 331, 337, 339, 341 show the direction of fluid flow. The filter/separator 334 diverts a portion for the plasma into line 336. That portion of the blood fluid which is not diverted to 336 is returned to the artery or vein 332. The divert plasma enters an emitter chamber 338 where the energy pattern is applied to the plasma. Said energy pattern may be millimeter wave, acoustic energy, light, etc. as describe throughout this disclosure. The treated plasma returns to the artery or vain through line 340. By way of illustration, but not limitation, 334, 336, 338 may be components of an artificial heart implant into the body.

In another embodiment, fluid is treated externally and independent of a body as it flows through tubing. In FIG. 14, tubing 350 carrying a fluid 353 flowing in the direction 354 has an emitter 358 implanted through the tubing wall so that the emitter tip 356 is in contact with the fluid 352. By way of illustration, but not limitation, said tubing 350 may be the fuel line of a vehicle, the water supply line to a faucet, the outlet of a water dispenser, an intravenous (IV) line, an implanted stint, etc. The emitter 358 is connected to a controller 362 by communications means 360 which may be, e.g., a wire, fiber optics cable, RF or other means. The controller 362 controls the type of energy, pattern of energy, application timing, duration, magnitude and/or other adjustable parameters. Additionally, a optional sensor 364 may be inserted into the tubing. Said sensor 364 may measure, e.g., the flow rate of the fluid 352 and/or the temperature of the fluid 352, the pH level of the fluid 352 and/or other measurable properties. Said sensor is connected to controller 362 by communication means 366 which may be, e.g. a wire, fiber optic cable, RF or other means.

In another embodiment (not shown), the emitter tip 356 of FIG. 14 is attached externally to the tubing wall 350. In this embodiment, the emitter tip 356 does not come into direct contact with the fluid 352.

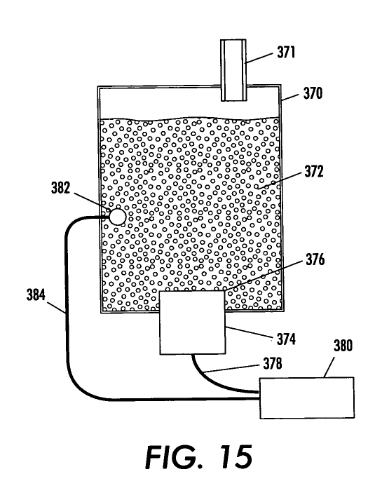

In another embodiment, see FIG. 15, the fluid 372 to receive the energy pattern treatment is preferably contained in a vessel 370 which has means 371 for removing and/or replenishing said fluid 372. By way of illustration and not limitation, said vessel 370 may be e.g., a hot water heater, a thermos or canteen, a coffee maker, an IV bag, a gasoline tank, etc. In one embodiment, emitter 374 has its emitting tip 376 in contact with fluid 372. In another embodiment (not shown) the emitter tip 376 is external to the vessel 370. In FIG. 15, the emitter 374 is connected to controller 380 via communication means 378 which may be, e.g., a wire, fiber optics cable, RF or other means. The controller 380 controls the type of energy, pattern of energy, application timing, duration, magnitude and/or other adjustable parameters. Additionally, a optional sensor 382 may be inserted into the vessel 370. Said sensor 382 may measure, e.g., the temperature of the fluid 372, the pH level of the fluid 372 and/or other measurable properties. Said sensor 382 is connected to controller 380 by communication means 384 which may be, e.g. a wire, fiber optic cable, RF or other means.

A Process for the Treatment of, Diseased Organisms

In yet another embodiment of the invention, a process for the treatment of disease, such as cancer is provided. Although the process is applicable to many different diseases, it will be described by reference to cancer for ease of simplicity of description.

The group of diseases commonly referred to as cancer in fact includes a highly diverse set of cell types that have, through a process of mutation, begun a process of unregulated proliferation. Since the accumulation of these mutations is a random process, the combination of mutations that ultimately result in a cancerous disease state varies widely. This complicates the process of disease treatment, as each protocol must be tailor-made to suit each different patient.

Physicians have long sought a treatment for cell proliferation diseases (such as cancer) that could be generalized for the treatment of all of these related maladies, avoiding this process of “tailoring making” a protocol that may involve invasive diagnostic techniques that can be uncomfortable for the patient and rely on conventional pathological analysis which is expensive, time-consuming and often is based on techniques that have variable accuracy.

One unique property of cancer cells is their ability, once in their fully transformed state, to become motile. This property is known to those of skill in the art as invasive and metastasis. Reference may be had, e.g. to U.S. Pat. No. 5,260,288 (“Method for inhibition of cell motility by sphingosine-1-phosphate and derivatives,”) that discloses that “cell motility is an important parameter defining various pathological processes such as inflammation, tumor invasion, and metastasis.”(See column 1, Line 57-60). The entire content of this United States patent are hereby incorporated by reference into this specification.

In one embodiment, describe more fully elsewhere in this specification, there is disclosed a process and device to influence the cell motility and cell division cycle of cancer and other diseased cells in order to slow or stop their proliferation and thereby slow the progression of the disease or affect a cure. As will be apparent to those skilled in the art, disease processes involving the control of cell proliferation include, but are not limited to restenosis of vascular and arteriole tissue following angioplasty or the introduction of a vascular stent, the development of excessive or unwanted scar tissue, thickening of ventricular walls in hypertrophic cardiomyopathy, angiogenesis of tumor masses, psoriasis, and other related disorders.

These disease processes are well described in the patent literature. Thus, by way of illustration and not limitation, reference may be had, e.g., to U.S. Pat. No. 6,417,338 (“Autotaxin: motility stimulating protein useful in cancer diagnosis and therapy”), the entire contents of which is hereby incorporated by reference into this specification. This patent states that “cell motility plays an important role in embryonic events, adult tissue remodeling, wound healing, angiogenesis, immune defense, and metastasis of tumor cells (Singer, 1986). In normal physiologic processes, motility is tightly regulated. On the other hand, tumor cell motility may be aberrantly regulated or autoregulated.”(see column 1, lines 29 to 34) There is a great clinical need to predict the aggressiveness of a patient's individual tumor, to predict the local recurrence of treated tumors and to identify patients at high risk for development of invasive tumors.”

Additionally, U.S. Pat. No. 6,844,184: (“Device for arraying biomolecules and for monitoring cell motility in real-time”), the entire contents of which is hereby incorporated by reference into this specification, reiterates the importance of cell motility in disease by stating that “When a cell is exposed to chemical stimuli, its behavior is an important consideration, particularly when developing and evaluating therapeutic candidates and their effectiveness. By documenting the reaction of a cell or a group of cells to a chemical stimulus, such as a therapeutic agent, the effectiveness of the chemical stimulus can be better understood. In particular, in the fields of oncology and cell biology, cell migration and metastasis are regularly considered. Typically, studies in these fields involve analyzing the migration and behavior of living cells with regard to various biological factors and potential anti-cancer drugs. Moreover, the resultant migration, differentiation, and behavior of a cell are often insightful towards further understanding the chemotactic processes involved in tumor cell metastasis. In addition, these studies can also provide insight into the processes of tissue regeneration, wound healing, inflamation, autoimmune diseases, and many other degenerative diseases and conditions”(see column 1, lines 36-53).

By way of further illustration, U.S. Pat. No. 6,844,184 discloses that “cell migration assays are often used in conducting these types of research. Commercially available devices for creating such assays are often based on or employ a Boyden chamber (a vessel partitioned by a thin porous membrane to form two distinct, super-imposed chambers). Also known as transwells, the Boyden chamber is used by placing a migratory stimulus on one side of a thin porous membrane and cells to be studied on the other. After a sufficient incubation period the cells may be fixed, stained, and counted to study the effects of the stimulus on cell migration across the membrane” (see column 1, lines 54-64).

Cell motility and invasion can be described experimentally, as is well known to those skilled in the art. Reference may be had to U.S. Pat. No. 5,260,288 (a method “ . . . for determining chemotactic cell motility and chemoinvasion . . . . ” Reference also may be had to U.S. Pat. No. 5,260,288 (see column 4, line 61 to column 5, line 5) which discloses a method that “ . . . can be performed using transwell plates with a polycarbonate membrane filter (pore size 8 μm) (Costar Scientific, Cambridge, Mass.). Aliquots, e.g., 50 μl, of an aqueous solution of MATRI-GEL (Collaborative Research, Bedford, Mass.) containing SPN-1-P or other inhibitor (e.g., 20 μg/ml for chemotactic motility assay or 200 μg/ml for chemoinvasion assay), is added to each well and dried overnight. The filter is then fitted onto the lower chamber plate. The lower chamber can contain conditioned medium (CM) (i.e., medium used for splenic stromal cell culture, and containing motility factor secreted by these cells), e.g., 0.6 ml, with or without SPN-1-P or other inhibitor.” The entire disclosure of each of these United States patents is hereby incorporated by reference into this specification.

By way of further illustration, U.S. Pat. No. 5,260,288 discloses that “[t]o the upper chamber is added, e.g., about 100 μl, of cell suspension (5×104 cells/ml for invasion assay, 5×105 cells/ml for motility assay), which is then incubated in 5% CO₂ at 37° C. for 70-72 hours (invasion assay) or 20 hours (motility assay). After incubation, cells remaining in the upper chamber are wiped off with a cotton swab, and cells which had migrated to the lower chamber side of the filter are fixed in methanol for 30 seconds and stained with 0.05% toluidine blue. The filter is removed, the stain is solubilized in 10% acetic acid (e.g., 0.1 ml for invasion assay, 0.5 ml for motility assay), and color intensity (optical density) is quantitated by ELISA reading at 630 nm. A schematic summary of this procedure is shown in FIG. 6. Using SPN-1-P, a linear relationship was observed between cell number and toluidine blue optical density (FIG. 7)” The entire disclosure of this United States patent is hereby incorporated by reference into this specification.

In order to interrogate aberrant cells, one may use the assay disclosed in U.S. Pat. No. 6,844,184, the entire disclosure of which is hereby incorporated by reference into this specification. This patent describes “an assay device or method that would allow further study of cell migration in response to various factors, including synergistic effects . . . .”.

U.S. Pat. No. 6,844,184 also discloses that “To study cell motility, either in response to a cell affecting agent, or random motility, it is desirable to be able to monitor cellular movement from a predefined “starting” position. To do this, cells must be placed, attached or immobilized upon a surface in such a manner that their viability is maintained and that their position is definable so that multiple interrogations or probing of cellular response (i.e. motility or lack thereof) may be performed. In previous methods concerning cell immobilization, cells often undergo a nonreversible immobilization. For example, cells have been immobilized by patterning cells on a self-assembled monolayer that has a protein tether that will “capture” the cell. Alternatively, cells have been immobilized via immunological reaction with antibodies, which themselves have been immobilized on the immobilization surface. Other methods of immobilization involve simply allowing cells to attach themselves to a suitable surface, such as glass or plastic, and then allowing them to migrate into adjacent areas” (see column 4, line 52 to column 5, line 3).

Other prior art references have also disclosed processes aimed at the prevention of cell motility aimed at the treatment of disease. Thus, e.g., U.S. Pat. No. 5,997,868 (“inhibition of scatter factor for blocking angiogenesis”), the entire contents of which is hereby incorporated by reference into this specification, discloses that “Angiogenesis is often associated with chronic inflammation diseases. Psoriasis is a common inflammatory skin disease characterized by prominent epidermal hyperplasia and neovascularization in the dermal papillae”(see column 7, lines 23 to 30).

U.S. Pat. No. 6,716,597(“methods and products for regulating cell motility”), teaches a method that “involves inducing a functional Ena/VASP protein in a mammalian cell in an effective amount for preventing cell migration.”(see column 5, lines 47 to 49). Further, it states that “the method involves administering to a subject having or at risk of developing a metastatic cancer a plasma membrane targeting compound in an effective amount for preventing cell migration in order to prevent tumor cell metastasis. In yet other aspects, the invention is a method for preventing or treating inflammatory disease in a subject” (see column 5, lines 51 to 57). The entire disclosure of each of these United States patents is hereby incorporated by reference into this specification.

By way of further illustration, U.S. Pat. No. 5,994,325: (methods and compositions based on inhibition of cell invasion and fibrosis by anionic polymers), the entire contents of which is hereby incorporated by reference into this specification, discloses “the discovery that biocompatible anionic polymers can effectively inhibit fibrosis, scar formation, and surgical adhesions. The invention is predicated on the discovery that anionic polymers effectively inhibit invasion of cells associated with detrimental healing processes, and in particular, that the effectiveness of an anionic polymer at inhibiting cell invasion correlates with the anionic charge density of the polymer.” Additionally, “the invention further provides compositions and methods to inhibit glial cell invasion, detrimental bone growth and neurite outgrowth.”

The prior art has disclosed devices and processes for influencing the behavior of cells by exposing them to light. Thus, e.g., Dr. Guenter Albrecht-Buehler reports that 3T3 cells can be influenced to move in the direction of a source of infrared light in the “Journal of Cell Biology” (Vol. 114, Num. 3, August 1991 pages 493-502). He states that: “using a specially designed microscope with an infrared spot illuminator we found that approximately 25% of 3T3 cells were able to extend pseudopodia towards single microscope infrared light sources nearby. If the cells were offered a pair of such light sources next to each other, 47% of the cells extended towards them.”

In this Albrecht-Buehler article in the Journal of Cell Biology (114:3, pages 493-502), the author describes the design of the Infrared Spot-irradiation Phase-Contrast Light Microscope (IRSIP) as follows: “the opaque center of the illumination annulus of commercial phase-contrast condensers would have blocked the incident infrared light beam. Furthermore, its glass lenses would have absorbed infrared light above wavelengths of X>2.5 Am (10). Therefore, we replaced the phase contrast condensor with a fiber optical illuminator in the shape of a ring (FIG. 1, 4); 5-cm ring diameter, 210 W Intralux, 6,000 illuminator (Volpi, AG, Schlieren, Switzerland) at such a distance below the microscope stage that the objective lens (FIG. 1, 2) imaged it onto its phase ring (FIG. 1, 5). In this way it was guaranteed that all the undiffracted light from the light source passed through the phase ring while the optical axis of the illuminator remained empty for the infrared light to pass freely along its length (FIG. 1, 6). A sapphire lens (5-mmdiam; 5-mm focal length; Melles Griot, Rochester, N.Y.) focused the infrared light into the chamber. Sapphire (AI203) was used because it transmits infrared light up to a wavelength of a=7 Am (11). 1b allow the light from the ring illuminator to pass, the sapphire lens was mounted in a transparent Plexiglass plate (FIG. 1, 3).”

In this article, Albrecht-Buehler elaborates the description of his device by stating that the Spot Illuminator is constructed with “Field Illumination Wavelengths. Based on our earlier investigations about the least perturbing field illumination spectrum for long-term observation of 3T3 cells (12), in most of the experiments we restricted the light for phase-contrast illumination to a small window between 600 and 700 run by combining a heat filter (BG38: Zeiss, Oberkochen, Germany) with a filter (RG 630; Corning Glass Division, Park Ridge, Ill.). In a special set of experiments (see Results) we used light of 510-560-nm wavelength (peak at 540 run) by combining a 540-nn interference filter with a CS3-70 absorption filter. All experiments were carried out in a darkened room to avoid effects of other wavelengths contained in the room light.”

The article then discloses that “Intensity. Using a cadmium sulfide photoconductive element as photometer and a Dewar flask filled with 400 ml of distilled water as a calorimeter we determined the normal illumination intensity in the range of wavelengths below X=2,000 ran to be I=0.48 mW/cm2. This intensity is N1/170a′ of the total solar irradiance of 80 mW/cm2 at sea level (11). THE SPOT ILLUMINATOR Monochromator The Beckman monochromator of a dismantled spectrophotometer (model 252; Gilford Instruments, Oberlin, Ohio) with a 20 W/6V halogen lamp (No. 778; General Electric, Co., Cleveland, Ohio) served as its infrared light source. Its spectral resolution at our normal setting of the slit width=1 nun was better than 10 nm as measured with a 3.5 mW HeNe Laser (Metrologic Instruments, Bellmawr, N.J.) which emits light at 633 t 1 run. Spot Size. To generate a well-defined outline of the irradiating spot, the light from the monochromator was sent through a small aperture (FIG. 1, 9) which was located 135 mm away from the sapphire lens which imaged it into the observation chamber (FIG. 1, p). The aperture was a standard platinum aperture of 100-Am diam used in scanning electron microscopes (E. F. Fullam, Inc., Latham, N.Y.). Its image (=spot size) had a diameter of 3.7 Am, corresponding to a spot area of AS=11 Am′. The glare generated by the gain control of the video camera made it appear larger on video images. Spot Temperature. Estimates show that the infrared light could not raise the temperature of the irradiated spot by more than 0.00001° C. (see Appendix). Incident Infrared Light Intensity. We used a charge-coupled device from a digitizing camera (EDC-1,000; Electrim Corp., Princeton, N.J.) to compare the intensity of the infrared light spot with the field illumination of the phase-contrast image at wavelengths of 600-700 run. We found that the spot intensity Is was approximately three times higher than the normal background, i.e., IS=1.5 mW/cm2 or − 1/50th of the intensity of sunlight.”

Albrecht-Buehler used the disclosed microscope to generate data about the response of cells to light in their environment, once again in his article in the Journal of Cell Biology (114:3, pp 493-502) stating that “Control Levels. We observed 83 individual cells for 1 h or longer in the infrared spot-irradiation phase-contrast microscope without using any infrared spot irradiation. We found that only three cells (4 t 2%) extended small lamellipodia at their tails. All others retracted the tail or kept it unchanged during the period of observation. Infrared Irradiation Experiments. In contrast, we found up to six times as many cells (24%; p<0.001 by t test) extending large lamellipodia towards the rear, if the base of their tail was exposed for 60 min to infrared spot irradiation with a sinusoidally oscillating amplitude at a frequency of 30/min. FIG. 3 shows an example of this inversion of cell polarity in the direction of an infrared spot-irradiated tail of a 3T3 cell. The action spectrum for this response (FIG. 4) was determined on the basis of 29-36 individual cell observations per selected wavelength. It showed a peak around 900 nm.”

In one preferred embodiment of this invention, also described elsewhere in this specification, infrared light is used to promote the migration of cells or cell appendages to a particular region. This migration can include, but is not limited to, angiogenesis, nerve axons, and myocytes.

In another article also by Dr. Guenter Albrecht-Buehler, “Cell motility and cytoskeleton” Vol. 32, pp. 299-304, he asserts that “3T3 cells respond differently to specific near-infrared signals than epithelial CV1 cells. Furthermore, signals with the same wavelength and energy changed the percentages of attracted and repelled 3T3 cells if their intensity modulation was altered. I have found this result in a 22 month long study which established a spectrum of motile responses of 781 individual 3T3 cells and 148 CV1 cells to the near-infrared emissions of microscopic, pulsating light sources using the infrared spot-irradiation phase-contrast microscope . . . . Since it seems to depend on the cell type and temoral pattern in which the light energy is emitted, it appears to imply the existence of a new kind of cellular information.” He further illustrates by stating that “If I used near-infrared of 800-900 nm wavelength with a pulsation frequency of 0.5 Hz about 28% of the test cells bridged distances of up to 60 micrometers with newly formed surface projections as they touched the light sources . . . . The cells essentially ignored light sources whose [sic.] intensity was constant.” Dr. Albrecht-Buehler then adds, “In addition to the general trends I observed in 3T3 cells a critical frequency range between 0.5 ad 1.0 Hz where “attraction” and “repulsion” were sharply frequency dependant: around 1.0 Hz nearly twice as many 3T3 cells were attracted as repelled, whereas I found the reverse situation at 0.7 Hz.”

In yet another 1998 article, in the journal “Cell motility and Cytoskeleton” Volume 40, pages 183-192, Albrecht-Buehler describes experiments in which infrared light was shown to “[reduce] the stability of radial microtubules around the centrosome.” In one preferred embodiment of this invention the application of light energy to the mitotic spindle of a dividing cancer cell prevents the completion of the cell division process.

In one preferred embodiment of this invention, pulsed infrared light is used to turn away invasive or migrating cells from an irradiated area. In another embodiment, light in the visual and ultraviolet spectrum is used. Each of these embodiments is described elsewhere in this specification.

In another preferred embodiment, non-cancer and cancer cells are cultured by methods routine to those skilled in the art, and exposed to light in the range of 660-1000 nanometers with varied pulse rates and wavelengths that were discovered to be capable of altering the progression of the cell cycle. This alteration could be a cessation or a signal to begin cell division. In another embodiment, light in the visual and ultraviolet spectrum is used.

In a preferred embodiment of this invention, an intravascular probe, such as that described in United States published patent application 20040039269: (“Use of ultraviolet, near-ultraviolet and near infrared resonance raman spectroscopy and fluorescence spectroscopy fro tissue interrogation of shock states, critical illnesses, and other disease states”), is used to deliver light energy to internal organs, vascular tissue and the like, with the goal of affected cell migration, cell division and other cellular processes as described in the above disclosure the entire contents of this United States patent application is hereby incorporated by reference into this specification. U.S. patent application 20040039269 claims “A tissue analysis method, comprising: interrogating a biological tissue with Raman spectroscopy and fluorescence spectroscopy to obtain spectroscopy results.”

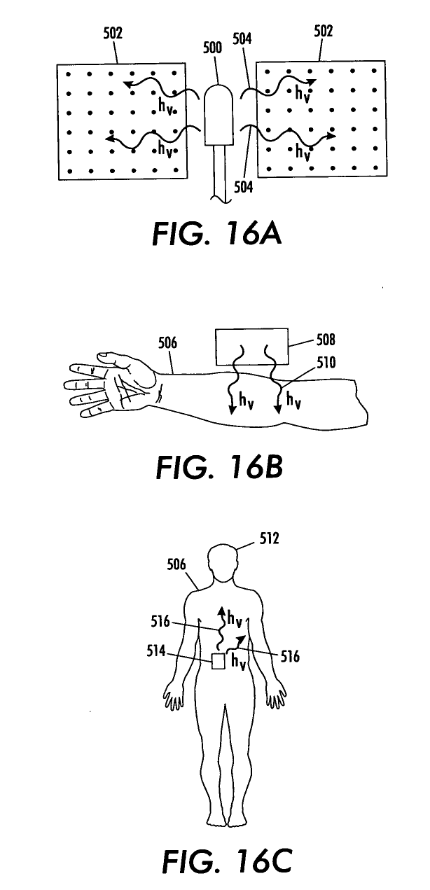

FIG. 16 describes one preferred process. FIG. 16A illustrates an intravascular probe 500 that is capable of delivering light, 504, of various wavelengths to tissues 502. These intravascular probes are well known to those skilled in the art. Reference may be had to U.S. Pat. No. 5,010,886: (“Medical probe assembly having combined ultrasonic imaging and laser ablation capabilities”). U.S. Pat. No. 5,010,886 claims “An intravascular probe assembly comprising: a distally located housing; optical fiber means having an end terminating within said housing for directing a beam of light along a first path generally parallel to a longitudinal axis of said housing; ultrasonic transducer means disposed within said housing for generating ultrasonic acoustic waves and for propagating said generated acoustic waves along a second path generally parallel to said housing longitudinal axis; means disposed in said first and second paths for redirecting said light beam and said acoustic waves from said first and second paths to respective redirected paths generally radially of said housing, and electrical connection means for electrically connecting said transducer means in series, said electrical connection means including; (i) means for establishing electrical connection between an outer periphery of said ultrasonic transducer means and said housing; (ii) two-lead cable means having a terminal end located within a proximal portion of said housing; (iii) a longitudinally extending channel defined in said housing and extending from a proximal location adjacent said terminal end of said two-lead cable means to a distal location adjacent a distal face of said transducer means; (iv) an electrically insulated wire disposed in said defined channel and having exposed proximal and distal ends at said proximal and distal locations of said defined channel, respectively; (v) an electrically conductive filler material which fills said channel and retains said wire therewithin; (vi) one of said leads of said two-lead cable means being electrically connected to said exposed proximal end of said wire at said proximal location of said channel; wherein (vii) said exposed distal end of said wire at said distal location of said channel is electrically connected to a distal face of said transducer means; and wherein (viii) another lead of said two-lead cable means is electrically connected to a proximal region of said housing, whereby said transducer means is series connected.” The entire disclosure of this United States patent is hereby incorporated by reference in this specification. FIG. 16B represents an external device 508 that delivers light energy, 510, to a patient 506. The use of this device is not limited to any appendage or target organ in particular. FIG. 16C represents an implantable version of a light emitting device 514 that can deliver light energy 516 to the patient 512.

In another section of this specification, various types and regimens of energy are delivered to a biological organism. It will be understood that any one or more of these energy protocols may be used with one or more of the devices of FIGS. 16A, 16B, and/or 16C.

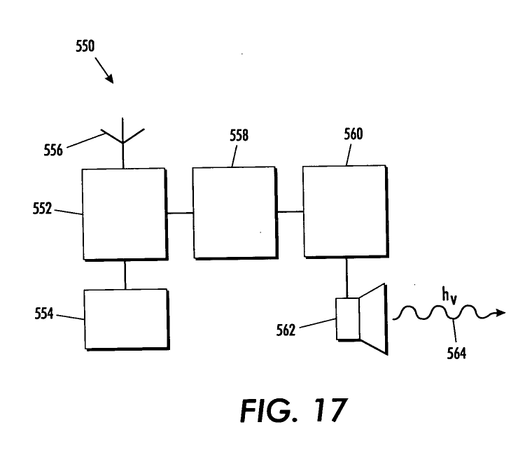

FIG. 17 is a diagram of another device 550 that may be used with one or more of such energy protocols. Referring to FIG. 17, and to the preferred embodiment depicted therein, it will be seen that device 550 may be used for the delivery of light energy 564 to a patient or cell system in vivo or in vitro. In one preferred embodiment, the device 550 is comprised of a main control unit 552 powered by an electrical power supply 554. An antenna 556, or antennae 556 allows for the telemetric reception of control signals from the exterior of the body, in the case of an implantable device, or other remote control in the case of external devices, and for the transmittance of information from the device to the user or caregiver using standard means of such radio transmission, known to those familiar with the art.

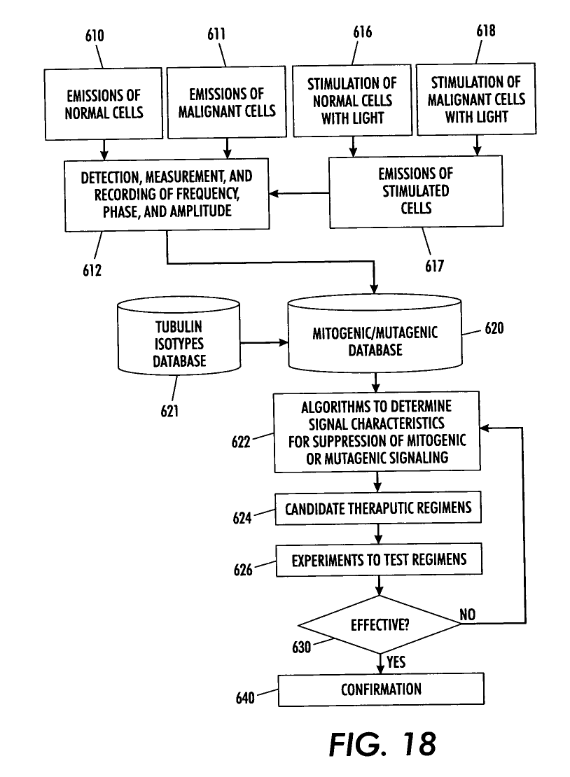

In one preferred embodiment, the control unit 552 uses information derived from the process described in FIG. 18 to operate solenoid 558. Solenoid 558 is preferably responsible for the temporal modulation of the signal, the pulse, as referred to above, produced by the transducer 560. Transducer 560 produces a frequency of light that is focused or dispersed (depending on the required regimen) by lens/reflector assembly 562 to produce the emission 564, the biologically active signal that is ultimately delivered to the organism or biological system.

In one embodiment the properties of the light emitted by one or more of the devices of FIGS. 16A, and/or 16B, and/or 16C, and/or 17 are described below.

In one embodiment, the wavelength is between about 601 and about 1200 nanometers. In another embodiment the wavelength is from about 390 to about 600 nanometers. In yet another embodiment, the wavelength is from about 200 to about 389 nanometers. In one aspect of these embodiments, regardless of the wavelength used, the light energy is pulsed into a biological sample (such as a tissue sample) at a 0.5 hertz pulse rate. In another aspect of this embodiment, the pulse rate is from about 0.55 hertz to about 0.7 hertz. In another aspect of this embodiment, the pulse rate is less than about 0.4 hertz and preferably is from about 0.1 to about ˜0.4 hertz. In another embodiment, when using one or more of the aforementioned wavelengths of light, the light is continuous.

A Process for Diagnosing and Treating Malignant Cells

FIG. 18 is a flow diagram of a preferred process for treating cells of biological organisms.

Referring to FIG. 18, and to the preferred embodiment depicted therein, electromagnetic emissions from normal and malignant cells (see elements 610 and 611, respectively), are detected, measured, compared, and stored in a database (see step 612 and element 620). The differences and frequency and phase-coherence relationships between the normal and malignant cells, both for individual patients and tubulin isotypes, are determined by analysis of this data 620. Based on this analysis, a plurality of different therapeutic regimens are then developed and tested (see steps 622, 624, 626, and 630) to determine the optimal regimens for particular patients and particular tubulin isotypes.

Referring again to FIG. 18, and in step 612 thereof, electromagnetic emissions, preferably in the range of 1 GHz to 1 teraHertz, from both normal cells 610 and malignant cells 611, are detected, measured, and recorded in step 612 to determine relevant measures of the biological signals such as, but not limited to, spectral density and phase coherence. Reference may be had to “ATIS Telecom Glossary 2000,” an outgrowth of the United States Federal Standard 1037 series, published by the Alliance for Telecommunications Industry Solutions and approved Feb. 28, 2001 by American National Standards Institute, Inc., which defines “spectral density” as “For a specified bandwidth of radiation consisting of a continuous frequency spectrum, the total power in the specified bandwidth divided by the specified bandwidth. Note: Spectral density is usually expressed in watts per hertz.” The ATIS Telecom Glossary 2000 also defines “phase coherence” as “The state in which two signals maintain a fixed phase relationship with each other or with a third signal that can serve as a reference for each.”

Spectral density and phase coherence of the emissions from normal cells 610 and malignant cells 611 are computed in step 612 and stored in database 620. Such detection and measurement can be accomplished by well-known electronic devices; e.g., spectrum analyzers and oscilloscopes. For a definition of spectrum analyzer, reference may be had to Sybil P. Parker “Concise Encyclopedia of Science and Technology, 3^{rd} Ed.” (McGraw-Hill: New York, N.Y.) 1994, page 1776. As stated in this reference a spectrum analyzer is “A device which sweeps over a portion of the radio-frequency spectrum, responds to signals whose frequencies lie within the swept band, and displays them in relative magnitude and frequency on a cathode ray tube screen. In essence, it is a superheterodyne receiver having a local oscillator whose frequency is varied cyclically, usually at the power line frequency.” Reference may also be had, e.g., to U.S. Pat. No. 4,598,247: (“Spectrum analyzer and analysis method for measuring power and wavelength of electromagnetic radiation.”) and U.S. Pat. No. 6,016,197: (“Compact, all optical spectrum analyzer for chemical and biologic fiber optic sensors”). The entire contents of these two United States patents are hereby incorporated by reference into this specification.

Optionally, and in one embodiment, Fast Fourier transform algorithms may be used with wideband signals to determine power spectral density of biological data by converting a signal in the time domain into data in the frequency domain, using either digital signal processors or the equivalent algorithms in software. Reference may also be had e.g., to U.S. Pat. No. 5,609,158: (“Apparatus and method for predicting cardiac arrhythmia by detection of micropotentials and analysis of all ECG segments and intervals”); U.S. Pat. No. 5,870,704: (“Frequency-domain spectral envelope estimation for monophonic and polyphonic signals”); U.S. Pat. No. 6,859,816: (“Fast Fourier transform method and inverse fast Fourier transform method”); U.S. Pat. No. 6,263,356: (“Fast fourier transform calculating apparatus and fast fourier transform calculating method”). The entire contents of these United States patents are hereby incorporated by reference into this specification.

As is known to those skilled in the art, such measurements of spectral density and phase coherence may be performed in a Faraday cage to attenuate artifacts from environmental sources. In addition, artifacts from electrical signals in the extreme low frequency (ELF) range, such as 60 Hz power line signals, may be attenuated by using battery-powered equipment, mu-metal shielding, common-mode-rejection circuits, and other methods.

Optionally, and in one embodiment, electromagnetic signals in the 100 to 1200 nanometer wavelength range are detected, measured, and recorded for normal cells 610 and malignant cells 611. One may detect, measure, and record spectral density and phase coherence for such signals by well-known devices; e.g., spectrophotometers, photomultipliers, and the like, or combinations thereof. Reference may be had e.g., to U.S. Pat. No. 6,714,304: (“Fourier transformation infrared spectrophotometer”); U.S. Pat. No. 6,549,795: (“Spectrophotometer for tissue examination”); U.S. Pat. No. 6,134,460: (“Spectrophotometers with catheters for measuring internal tissue”); U.S. Pat. No. 5,779,631: (“Spectrophotometer for measuring the metabolic condition of a subject”). The entire contents of these United States patents are hereby incorporated by reference into this specification. A detection device for detecting energy emissions from biological cells is also disclosed in published U.S. patent application 2003/0013094: (“Hybrid nucleic acid assembly”), the entire contents of which is hereby incorporated by reference into this specification.

By way of yet further illustration, one may use the processes disclosed in published U.S. patent 2003/0013094 (hybrid nucleic acid assembly), the entire disclosure of which is hereby incorporated by reference into this specification. This published patent application discloses “a process for measuring DNA conductivity (of electrons, photons, and vibration) while such DNA is undergoing its normal processes (such as transcription or replication) in substantially its normal environment . . . ” using “a hybrid nucleic acid assembly comprised of a partially denatured double strand of nucleic acid, a first probe attached to a proximal end of such strand, and a second probe attached to a distal end. Each of the first and second probes is comprised of a conductive fiber.”

Published U.S. patent application 2003/0013094 also discloses that “The energy transmitted may be light energy, either in the form of waves and/or particles, at various frequencies, wavelengths, or combinations thereof.” With such a device, “one can determine when there is any aberrant condition with such DNA that would affect such current flow, and/or one can determine when normal DNA processes (such as transcription or replication) are occurring. Reference data can be generated as to the current flows normally existing during these events, and such data can be correlated with readings taken from the DNA when it is in a substantially in vivo environment.”

Published U.S. patent application 2003/0013094 also discloses that “The electrical properties of DNA strand 72 will vary depending upon its geometry and chemical composition. These characteristics will, in turn, vary when events such as protein binding, transcription, replication, denaturation, and the like occur. Thus, the circuit 100 may be used to determine when a particular strand 72 of DNA is undergoing such an event and/or whether a particular strand of DNA 72 evidences an aberrant behavior or composition or geometry which affects such electrical characteristics.”

Optical phase coherence may alternatively be measured by means of such techniques and devices as, for example, laser diodes in combination with interferometers. Reference may be had e.g., to World Intellectual Property Organization patent WO05001445A2: (systems and methods for phase measurements) which states that “Preferred embodiments of the present invention are directed to systems for phase measurement which address the problem of phase noise using combinations of a number of strategies including, but not limited to, common-path interferometry, phase referencing, active stabilization and differential measurement. Embodiment are directed to optical devices for imaging small biological objects with light. These embodiments can be applied to the fields of, for example, cellular physiology and neuroscience. These preferred embodiments are based on principles of phase measurements and imaging technologies. The scientific motivation for using phase measurements and imaging technologies is derived from, for example, cellular biology at the sub-micron level which can include, without limitation, imaging origins of dysplasia, cellular communication, neuronal transmission and implementation of the genetic code. The structure and dynamics of sub-cellular constituents cannot be currently studied in their native state using the existing methods and technologies including, for example, x-ray and neutron scattering. In contrast, light based techniques with nanometer resolution enable the cellular machinery to be studied in its native state. Thus, preferred embodiments of the present invention include systems based on principles of interferometry and/or phase measurements and are used to study cellular physiology. These systems include principles of low coherence interferometry (LCI) using optical interferometers to measure phase, or light scattering spectroscopy (LSS) wherein interference within the cellular components themselves is used, or in the alternative the principles of LCI and LSS can be combined to result in systems of the present invention.”

Referring again to FIG. 18, and in steps 616 and 618 thereof, normal cells and malignant cells are stimulated by brief exposure to light in multiple wavelength increments throughout the 100 to 1200 nanometer range. Stimulated emissions from the normal and malignant cells are then detected and measured in step 617 to determine their degree of coherence, as described by Popp, F. A. and Li, K. H. in “Hyperbolic relaxation as a sufficient condition of a fully coherent ergodic field” in Recent Advances in Biophoton Research (F. A. Popp, K. H. Li and Q. Gu, eds.), pp. 47-58, World Scientific, Singapore. These data are stored in database 620.

The aforementioned steps (shown in 610, 611, 612, 616, 617, 618) are also repeated to determine the signature of electromagnetic radiation for normal and malignant cells for each isotope in the database of tubulin isotypes in step 621. These tubulin isotypes are described in applicants' copending United States patent application U.S. Ser. No. 10/923,615, (filed on Aug. 20, 2004), the entire disclosure of which is hereby incorporated by reference into this specification.

The signature (unique patterns) of the spectral density and phase coherence of the electromagnetic radiation for each group of normal and malignant cells is then determined by computational algorithms (which can be executed in either hardware or software) known to those skilled in the art, based on an analysis of the normal and malignant electromagnetic radiation and other data in the database.

Referring again to FIG. 18, and in step 622 thereof, based on this signature, as well as information gathered from the patient using conventional means that include, but are not limited to, a characterization of the predominant tubulin isotypes 621 present in the tumor cells of the patient in question, one or more algorithms 622 (which can be executed in either hardware or software) generate candidate therapeutic frequency and phase regimens 624 that are most effective in suppressing mitogenic or mutagenic signaling for malignant cells, using electromagnetic emissions with a plurality of pulse modulations, amplitude modulations, frequency modulations, phase modulations, pulse trains, and combinations of one or more these techniques.

Referring again to FIG. 18, candidate therapeutic frequency and phase regimens are determined that are most effective as electronic countermeasures (ECM). Reference may be had to ATIS Telecom Glossary 2000, which defines “electronic countermeasures” as “That division of electronic warfare involving actions taken to prevent or reduce an enemy's effective use of the electromagnetic spectrum.” Reference may also be had, e.g., to U.S. Pat. No. 6,492,931: (“Electronic countermeasures system and method”), which states, “Electronic countermeasures system for aircraft protection against enemy attack, has decoy which receives mixed signal from mixer through antenna, and modifies mixed signal for transmission”; U.S. Pat. No. 6,297,762: (“Electronic countermeasures system”), which is an “adaptive interferometer for tracking radar used in aircraft, supplies equal amplitude antiphase jamming signals to antennas when phase coincidence is judged between received signals”, and U.S. Pat. No. 4,112,300: (“Infrared electronic countermeasures”). The entire contents of these United States patents are hereby incorporated by reference into this specification.

In one embodiment, after a measurement is made of the emission(s) from the normal cells and/or the cancer cells, phase-cancellation signals are sent out to selectively confuse or incapacitate the cancer cells. Thus, e.g., one may use real-time phase cancellation, as known to those skilled in the art. Phase cancellation is achieved by transmitting an inverse (180 degrees out of phase) signal at the same frequency as a detected mitogenic or mutagenic signal. As a result, the mitogenic or mutagenic signal may be attenuated or blocked.

For example, mitogenic or mutagenic signals may be blocked using electronic countermeasures techniques such as, for example, electromagnetic radiation (at microwave or optical frequencies) that is modulated with high levels of noise (“jamming”) at target-sensitive frequencies or ranges of frequencies, or at specific power levels, or by specific pulse trains, or at specific phase regimens, or by synchronizing with mitogenic signals, or by using phase cancellation with mitogenic signals, or by using pulse trains that confuse mitogenic or mutagenic signals, or by using pulse trains that confuse by signalling completion of an event such as mitosis, or by any combination of these tactics, or by using a plurality of other electronic countermeasures techniques that are well known to those skilled in the art.

The candidate therapeutic regimens 624 are then executed in designed experiments 626 seeking to determine the optimal methods of entraining orderly cell division and appropriate coherence in electromagnetic energy emitted by cells. The effects of the candidate regimens are then measured, using standard techniques for the assessment of tumor mass growth, regression and remission known to those of skill in the art. In step 630 of such designed experimentation, a specific regimen that produces the desired therapeutic result for the patient, if successful, is determined and confirmed in final step 640.

A Light Emitting Coating for a Stent

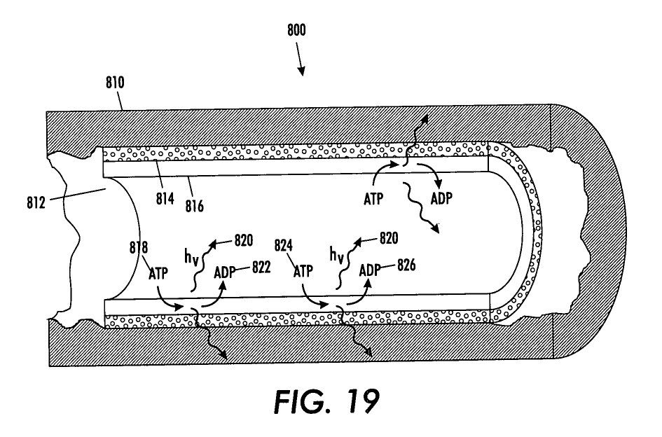

FIG. 19 is a schematic illustration of a coated stent 800 coated with a light emitting coating 810 for preventing restenosis.

Referring to FIG. 19, and in the preferred embodiment depicted therein, there is shown a cross sectional diagram of a blood vessel 810 that is being held open by a vascular stent 812. The stent is preferably constructed of a metal or plastic material 814 with a coating 816 disposed thereon. Coating 816 preferably contains a chemiluminescent material that converts sources of chemical energy, such as, e.g., ATP 818, available in the circulation, into a reduced form, ADP 822 and, in the process, produces light, 820. Chemiluminescent materials are well known to those skilled in the art. Reference may be had to U.S. Pat. No. 6,653,147: (“Apparatus and method for chemiluminescent assays”); U.S. Pat. No. 5,936,070: (“chemiluminescent compounds and methods of use”); U.S. Pat. No. 5,672,478: (“methods of use for and kits containing chemiluminescent compounds”). The entire contents of these United States patents is hereby incorporated by reference into this specification.

Referring again to FIG. 19, the chemical energy source, generically labeled 824, is not exclusively ATP but could be NADPH, glucose, pyruvate, or other available chemical energy sources. The reduced or other product of this reaction is generically referred to as P in FIG. 19 and is identified with numeral 826. This chemical reaction results in the release of light 820. In one preferred embodiment, the wavelength of the produced light is in the near-infrared part of the spectrum, i.e., about 800 to about 1200 nanometers, and interacts with the cells of the smooth muscle layer 810 of the vessel wall to inhibit cell migration into the interior of the stent, a process called restenosis. In another preferred embodiment, the wavelength of light released during this reaction is in the visible part of the spectrum, i.e., about 400 to about 800 nanometers.

In one preferred embodiment, described elsewhere in this specification, the wavelength of the light used is a wavelength determined a process described elsewhere to be toxic to cancer cells.

A Process for Treating Congestive Heart Failure

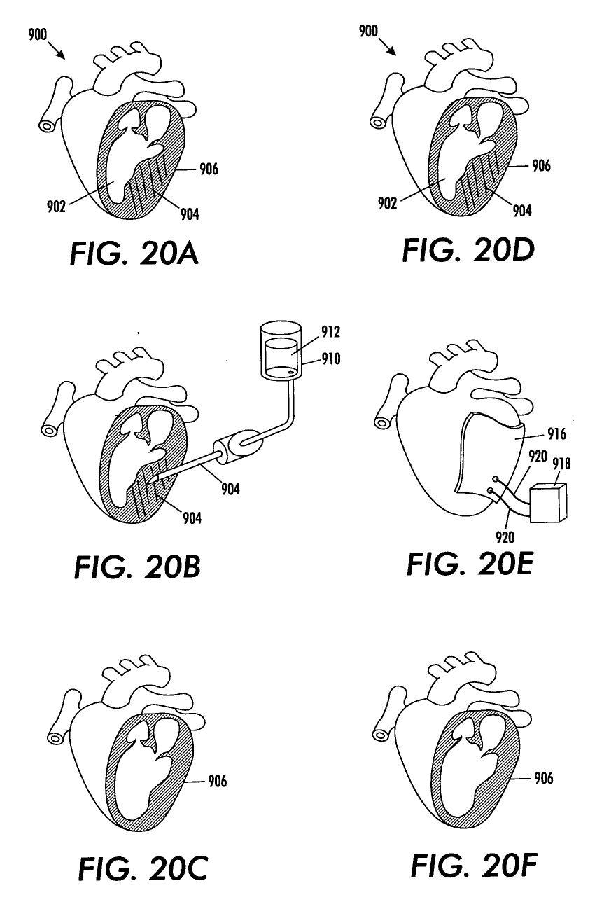

FIG. 20 is a schematic diagram of an process for treatment of congestive heart failure.

Referring to FIG. 20. There is shown a cross-sectional diagram of a human heart 900 with the left ventricle 902 partially occluded by hypertrophic cardiomyopathy 904 of the ventricle wall 906 of the heart. Hypertrophic cardiomyopathy, as is well known to those skilled in art, is a disease caused by the disorganized and inappropriate proliferation of cells of the wall 906 of the left ventricle 902 and leads to a debilitating and potentially fatal condition commonly known as congestive heart failure. FIG. 20A shows the diseased heart 900 before intervention. FIG. 20B shows the hypertrophic region 904 of the heart being penetrated with a syringe 908 and injected with an inoculant 912 contained in a vessel 910. Elements 908 and 910 are not necessarily drawn to scale. The inoculant 912 contains a quantity of genetically engineered, reproduction incompetent, viruses capable of infecting the hypertrophic cells. This virus preferably contains a gene capable of expressing a protein in a eukaryotic system that is chemiluminescent; that is, it uses energy sources in the cell to produce light. In one preferred embodiment, the wavelength of the produced light is in the near-infrared part of the spectrum, i.e., about 800 to about 1200 nanometers, and interacts with the cells of the ventricular wall 906 and the aberrant hypertrophic cells 904, to inhibit cell migration and cell division into the interior of the left ventricle 902. In another preferred embodiment, the wavelength of light released during this reaction is in the visible light part of the spectrum, i.e., about 400 to about 800 nanometers. This reduction of cell proliferation and migration results in the restoration of the ventricular wall 906 to its normal, non-diseased size, restoring function and successfully treating the congestive disease state.

In another preferred embodiment of this invention, and with reference to FIG. 20D, a hypertrophic heart is fixed with a patch or cover, 916 (see FIG. 20E). This patch is connected to control and power unit 918 by wires 920, or, in another preferred embodiment, is self powered by chemical energy sources in the thoracic or by the kinetic motion of the heart. This patch is capable of the emission of light energy (as described in previously in this disclosure) in order to slow or prevent the enlargement of the hypertrophic growth, 904, of the ventricle wall, 906. In an additional preferred embodiment, the energy emitted by the patch includes sources of energy other than light energy. In another preferred embodiment, the patch gathers information from the heart and is able to telemetrically communicate this information to a clinician caring for the patient. This monitoring of the cardiac function includes, but is not limited to, the collection of sound information. As will be apparent to those skilled in the art, hypertrophic disease causes a change in, among other measurable conditions that will be apparent to those skilled in the art, the sound of the blood moving through the heart chambers and the valves between them. By constantly monitoring the sound, or other conditions, of the heart, heart failure, murmurs or infarction or other conditions may be detectable in the early stages, before a potentially fatal cardiac crisis is underway. Control unit 918 could then inform the clinician, patient or emergency medical team that acute medical intervention is required. This telemetric communication with heath care providers includes, but is not limited to wireless devices and technologies such as “Bluetooth,” cell phone or satellite locating and warning systems known to those of skill in the art.

A Device for Interrogating Cellular Components

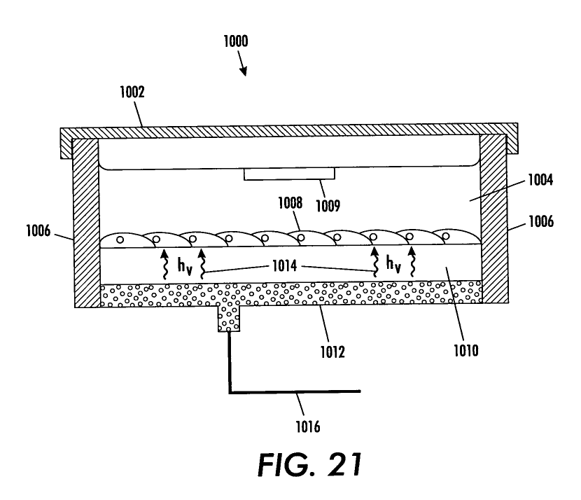

FIG. 21 is a schematic illustration of a device 1000 for interrogating cellular components. The cellular components to be interrogated include, e.g., centrioles, microtubules, actin and actin-containing structures, tubulin and tubulin-containing structures, proteins of membrane or the cytoplasm or the mitochondria, and the like.

Referring again to FIG. 21, and to the preferred embodiment depicted therein, the cellular components are disposed in a solution containing gelatin, agarose, or other solid or semi-solid media capable of supporting a eukaryotic cell monolayer, such as monolayer 1008. The eukaryotic cell monolayer may be, e.g., a monolayer transformed or non-transformed immobilized cell lines, or cells of primary culture. In one embodiment, eukaryotic cell monolayer is 3T3 cells, COS-1 cells, C6 cells, and the like.

In the embodiment depicted, the monolayer 1008 is disposed on the media 1010. A light emitting device 1012 is preferably disposed below the monolayer 1008, and it is adapted to emit one or more of the radiations described elsewhere in this specification.

In the preferred embodiment depicted, and referring again to FIG. 21, the light source 1012 is connected via line 1016 to a controller (not shown) that is adapted to control the emission produced by light source 1012 in a pattern that is either predetermined and/or is determined by one or more measured parameters.

Lid 1002 is configured so as to ensure an optically sterile environment; it is preferably opaque, neither allowing light to enter or leave. As used herein, the term light with a wavelength of from about 200 to about 1200 nanometers.

In the preferred embodiment depicted, the device 1000 is preferably shielded from radio frequency radiation by Mu metal shields 1006.

Referring again to FIG. 21, as light energy 1014 is emitted from light source 1012, it travels through the media 1010 and thereafter contacts the cellular components (not shown) disposed in media 1010. Without wishing to be bound to any particular theory, applicants believe that the cellular components disposed in media 1010, upon being contacted with light energy 1014, emit harmonics thereof. Thus, e.g., it the light source radiates energy 1014 with a wavelength of 800 nanometers, a second harmonic (400 nanometers) and a third harmonic (200 nanometers, and/or other harmonics) are emitted.

In one preferred embodiment, it is preferred to generate harmonics with a wavelength of from 200 to 400 nanometers. In this embodiment, one thus would utilize a light source 1012 that produced light with a wavelength of 400 to 800 nanometers.

After light energy 1014 has been emitted from the light source 10-12, one can observe the effect of such light energy 1014 (and/or of the harmonics it creates) upon the cell monolayer 1008. One can remove the lid 1002 and observe whether the cells have proliferated, and/or been killed, and/or moved. In one embodiment, camera 1009 continually monitors the effects of the radiation 1014 upon the cell monolayer and transfers this information by a telemetric link (not shown) to the controller (not shown).

As will be apparent, the device 1000 allows one to determine the effects, if any, upon cellular health of various light regimens. Some of these are discussed elsewhere in this specification.

In one preferred embodiment, the light regimen in question preferentially kills cancer cell and/or preferentially stops the cell division of cancer and/or preferably stops the motility of cancer cells.

In one embodiment, the cell monolayer 1008 is a cell monolayer derived from cancer cells taken from a patient. As will be apparent, one can determine, for these particular cells in question, which light energy regimen is most efficacious in treating such cancer cells. Thereafter, one can implant a device, such as the device depicted in FIG. 16C, in the patient and program the device to direct the appropriate light therapy to the tumor in question. As will be apparent, cells contained in a hypertrophic left ventricle (see FIG. 20) may also be interrogated with this device 1000 so as to find a signal that is non-proliferative for hypertrophic versus normal myocytes. These frequencies can then be used in the device of FIG. 20E.

Treatment of the Biological Material with Solitons and/or Phonons

In one preferred embodiment, the biological material referred to elsewhere in this specification is treated with either solitons and/or phonons.

As is known to those skilled in the art, a soliton is an isolated wave that propagates without dispersing its energy over larger and larger regions of space and whose nature is such that two such objects emerge unchanged from a collision. Reference may be had, e.g., to page 1770 of the “McGraw-Hill Dictionary of Scientific and Technical Terms,” Fourth Edition (McGraw-Hill Book Company, New York, N.Y., 1989). Reference may he had, e.g., to U.S. Pat. No. 5,157,744 (soliton generator), U.S. Pat. No. 5,473,458 (soliton data transmission using non-soliton transmitter), U.S. Pat. No. 5,477,375 (optical soliton generator), U.S. Pat. No. 5,508,845 (quasi-soliton transmission system), U.S. Pat. No. 5,523,874 (optical soliton pulse transmission system), U.S. Pat. No. 6,130,767 (method and apparatus for conditioning optical solitons), U.S. Pat. No. 6,134,038 (optical signal for a soliton optical transmission system), U.S. Pat. No. 6,222,669 (optical partial regeneration of solitons), U.S. Pat. No. 6,342,962 (optical system for transmitting data in soliton format), U.S. Pat. No. 6,441,939 (device and method for regenerating a train of solitons), U.S. Pat. No. 6,449,408 (soliton pulse generator), and the like. The entire disclosure of each of these United States patents is hereby incorporated by reference into this specification.

It is to be understood that the aforementioned description is illustrative only and that changes can be made in the apparatus, in the ingredients and their proportions, and in the sequence of combinations and process steps, as well as in other aspects of the invention discussed herein, without departing from the scope of the invention as defined in the following claims. Moreover, it is to be understood that maintaining the proper physiology of the heart and liver and carbohydrate metabolism and other organs and tissues have been used as examples of the application of the invention, and that many other diseases and disorders can be approached with this invention without departing from the scope of the invention as defined in the following claims.

1. A process for treating a biological organism, said process comprising the steps of:
(a) measuring an emitted energy emitted by said biological organism as said biological organism is being stimulated by a therapeutic agent;
(b) stimulating said organism sequentially, one at a time, with a predetermined plurality of stimulation energies;
(c) identifying and selecting, from said plurality of stimulation energies in step (b), a matching energy, said matching energy having stimulated said organism to emit the same said emitted energy as said therapeutic agent in step (a); and
(d) directing, with an energy emitting device, said matching energy to said organism according to a predetermined protocol.

2. The process as cited in claim 1, wherein said energy emitting device is operationally connected to a programmed controller.

3. The process as cited in claim 2, wherein, during step (d) a condition of said biological organism is sensed with a sensing device operationally connected to said programmed controller.

4. The process as cited in claim 3, wherein said emitted energy and said matching energy are comprised of electromagnetic energy.

5. The process as cited in claim 4, wherein said matching energy is 180 degrees out of phase with said emitted energy.

6. The process as cited in claim 3, wherein said biological organism is a living mammal.

7. The process as cited in claim 6, wherein said energy emitting device is implanted in said living mammal.

8. The process as cited in claim 7, wherein said sensing device is implanted in said living mammal.

9. An apparatus for treating a biological organism, said apparatus comprising:
a first energy emitting module for delivering a first energy to said biological organism, said first energy emitting module being implanted in said biological organism;
a second energy emitting module for delivering a second energy to said biological organism, said second energy emitting module being removably inserted into said biological organism.

10. The apparatus as recited in claim 9, wherein said biological organism is a living mammal.

11. The apparatus as recited in claim 10, wherein said first energy and said second energy are electromagnetic waves with a wavelength of from about 601 to about 1200 nanometers.

12. The apparatus as recited in claim 11, wherein said first energy and said second energy are delivered to said biological organism as pulses at a pulse rate of about 0.5 hertz.

13. The apparatus as recited in claim 11, wherein said first energy and said second energy are delivered to said biological organism as pulses at a pulse rate of from about 0.55 to about 0.7 hertz.

14. The apparatus as recited in claim 11, wherein said first energy and said second energy are delivered to said biological organism as pulses at a pulse rate of from about 0.1 to 0.4 hertz.

15. The apparatus as recited in claim 11, wherein said first energy and said second energy are delivered to said biological organism as continuous waves.

16. The apparatus as recited in claim 10, wherein said first energy and said second energy are electromagnetic waves with a wavelength of from about 390 to about 600 nanometers.

17. The apparatus as recited in claim 16, wherein said first energy and said second energy are delivered to said biological organism as pulses at a pulse rate of about 0.5 hertz.

18. The apparatus as recited in claim 16, wherein said first energy and said second energy are delivered to said biological organism as pulses at a pulse rate of from about 0.55 to about 0.7 hertz.

19. The apparatus as recited in claim 16, wherein said first energy and said second energy are delivered to said biological organism as pulses at a pulse rate of from about 0.1 to 0.4 hertz.

20. The apparatus as recited in claim 16, wherein said first energy and said second energy are delivered to said biological organism as continuous waves.

21. The apparatus as recited in claim 10, wherein said first energy and said second energy are electromagnetic waves with a wavelength of from about 200 to about 389 nanometers.

22. The apparatus as recited in claim 21, wherein said first energy and said second energy are delivered to said biological organism as pulses at a pulse rate of about 0.5 hertz.

23. The apparatus as recited in claim 21, wherein said first energy and said second energy are delivered to said biological organism as pulses at a pulse rate of from about 0.55 to about 0.7 hertz.

24. The apparatus as recited in claim 21, wherein said first energy and said second energy are delivered to said biological organism as pulses at a pulse rate of from about 0.1 to 0.4 hertz.

25. The apparatus as recited in claim 21, wherein said first energy and said second energy are delivered to said biological organism as continuous waves.

26. The apparatus as recited in claim 9, further comprising a sensing device for sensing a condition of said biological organism, said sensing device communicates with a programmable controller, said programmable controller also communicates with said first and said second energy emitting modules, for respectively varying, in response to said sensing device, said first and said second energy.

27. The apparatus as recited in claim 26, wherein said sensing device is externally connected to said biological organism.

28. The apparatus as recited in claim 26, wherein said sensing device is implanted in said biological organism.

29. The apparatus as recited in claim 26, wherein said programmable controller communicates remotely with said sensing device and said first and said second energy emitting modules with electromagnetic waves.

30. The apparatus as recited in claim 29, wherein said biological organism is a living mammal.

31. An apparatus for treating a biological organism, said apparatus comprising:
a first energy emitting module for delivering a first energy to said biological organism, said first energy emitting module being implanted in said biological organism;
a second energy emitting module for delivering a second energy to said biological organism, said second energy emitting module being externally connected to said biological organism.

32. The apparatus as recited in claim 31, wherein said biological organism is a living mammal.

33. The apparatus as recited in claim 32, further comprising a sensing device for sensing a condition of said biological organism, said sensing device communicates with a programmable controller, said programmable controller also communicates with said first and said second energy emitting modules, for respectively varying, in response to said sensing device, said first and said second energy.

34. The apparatus as recited in claim 33, wherein said sensing device is externally connected to said biological organism.

35. The apparatus as recited in claim 33, wherein said sensing device is implanted in said biological organism.

36. The apparatus as recited in claim 33, wherein said programmable controller communicates remotely with said sensing device and said first and said second energy emitting modules with electromagnetic waves.
